(19) United States
(12) Patent Application Publication
(10) Pub. No.: US 2016/0258944 A1
(43) Pub. Date: Sep. 8, 2016

(54) Integrated Microreactor Array System

(71) Applicant: Peter Wiktor, Phoenix, AZ (US)

(72) Inventor: Peter Wiktor, Phoenix, AZ (US)

(21) Appl. No.: 15/054,230

(22) Filed: Feb. 26, 2016

Related U.S. Application Data
(60) Provisional application No. 62/127,156, filed on Mar. 2, 2015.

(51) Int. Cl. G01N 33/543

(52) U.S. Cl. CPC G01N 33/54386

(57) ABSTRACT

A microreactor array system includes an array of microreactors, and a sealing membrane having a first surface and an opposite second surface, the sealing membrane configured to movably seal the array of microreactors. The microreactor array system further includes a reagent gap providing a fluid path between the array of microreactors and the second surface of the sealing membrane when the second surface of the sealing membrane is in spaced relationship with the array of microreactors. The microreactor array system further includes an injector for delivering a reagent into the reagent gap, an applicator for directing a working fluid against the first surface of the sealing membrane, and a detector.

### CROSS-REFERENCE TO RELATED APPLICATIONS

This application claims priority from U.S. Patent Application No. 62/127,156 filed Mar. 2, 2015.

### STATEMENT REGARDING FEDERALLY SPONSORED RESEARCH

This invention was made with government support under grant number R42 GM106704 awarded by the National Institutes of Health. The government has certain rights in the invention.

### BACKGROUND

The present disclosure relates generally to devices, systems and methods for controlling micro-reactions and, in particular, to devices, systems and methods for controlling micro-reactions in high throughput parallel assays.

Human genomes have approximately 20,000 genes that code for proteins. While this represents only about 2% of the entire genome, much of the remaining 98% (i.e., “noncoding DNA”) provide important biological functions including transcription and translation regulation of the protein coding sequences. Consequently a single gene may code for multiple different alternative splicings depending on regulation signals. Function of these proteins is further differentiated by various post-translational modifications (PTMs) such as glycosylation or phosphorylation. The result is that the human proteome may have over a million different protein isoforms and PTMs. Adding to this complexity is the microbiome that includes the vast number of symbiotic and pathogenic microorganisms that cohabitate with humans—each with its own genome and proteome. Further, there is the antibodyome, or all of the different antibodies produced by the immune system in response to exogenous antigens or to endogenous antigens due to cancer or autoimmune disease.

The molecules of the genome, proteome, microbiome and antibodyome interact in complex ways to regulate biological functions in health and disease. Correlating molecular interactions with clinical data may involve screening large numbers of patient samples to get statistically meaningful results. This may rely on high-throughput detection systems that can simultaneously detect and quantify thousands of molecular biomarkers with high sensitivity and selectivity. However, one challenge facing the emerging field of biomarkers is that many relevant biomarkers are present at very low abundance in human samples.

The life sciences have transitioned from one experiment at a time; to multiplexed experiments in 96-well plastic microtiter plates; to a million simultaneous experiments for gene expression analysis on microarrays; and to around a billion for next generation DNA sequencing. Generally, these high-throughput experiments are based on molecules tethered to a surface. However, chemical reactions in living cells typically involve untethered, free floating molecules in aqueous solutions. Many different biochemical reactions may occur simultaneously depending on cell type, cell cycle or external stimuli. Accordingly, it may be useful to provide high-throughput experimental systems to simultaneously study thousands of biochemical reactions involving untethered, intermediate, free floating, molecular compounds.

Current applications for high-throughput molecular detection assays include gene expression profiling, genotyping, DNA profiling, polymerase chain reaction (PCR), DNA sequencing, immunoassays, high-throughput screening (HTS), in vitro transcription and translation (IVTT) and high-throughput cell analysis. Various different types of high-throughput systems are often used to carry out these various assays depending on specific applications such as basic biology research, environmental monitoring, select agents detection, forensics, proteomics, functional genomics, epidemiology, drug discovery, clinical molecular diagnostics or personalized medicine.

Chemical reactions, in living organisms, involve free floating molecules in liquid water. Therefore experiments to study these reactions, in the laboratory, are typically performed in aqueous solutions. For some experiments, molecules may be chemically bound to a surface. Microarrays, for example, include molecular targets bound to a surface in a pattern of rows and columns. The microarray surface may be flooded with a sample containing various different probe molecules that bind to the probes. After washing, the target molecules are labeled, detected and identified based on where they bind to the targets in the array. However other types of experiments involve unbound, free floating molecules.

For example, enzyme-linked immunosorbent assay (ELISA), uses chromogenic or fluorogenic substrates in solution that react with enzymes bound to a surface. These substrates are physically contained together with the enzymes during an experiment. However ELISA is typically limited to testing only a handful of analytes at a time, although higher throughput systems have been investigated.

Similarly, PCR amplifies DNA template molecules freely floating in a buffer solution. However, PCR, real-time PCR and quantitative real-time PCR (qPCR) is currently typically limited to detecting at most only a few hundred analytes at a time.

Protein expression in living cells involves untethered intermediate molecules such as mRNA, enzymes, ribosomes, amino acids and polypeptides. Proteins can also be expressed outside of living cells by subjecting genomic DNA to cell-free IVTT reagent. This is the process used for nucleic acid programmable protein arrays (NAPPA) to express unique proteins from plasmid cDNA containing their full length genes. Proteins are expressed and captured in situ in a microarray format at the time of assay. The microarrays are used to assay thousands of protein interactions simultaneously. NAPPA may be used to discover autoantibody biomarkers correlated to specific diseases and to detect antibodies to pathogens. To preserve protein function, assays using NAPPA are typically done within hours of expressing fresh proteins without ever allowing them to dry out. By comparison, conventional protein microarrays are based on purified proteins printed from frozen stock and then stored possibly for months before assay.

In situ protein expression for NAPPA is typically carried out on flat microscope slides by flooding the entire microarray surface with IVTT reagent. Spot to spot diffusion currently limits NAPPA density to ˜2,500 protein spots per slide. Density may be increased by expressing proteins in an array of micro-reaction chambers (i.e., microreactors, microwells or nanowells).

Therefore, given the above, there is a need for systems and methods for controlling micro-reactions in high throughput parallel assays.

### SUMMARY

According to one embodiment of the present disclosure, a microreactor array system includes an array of microreactors, and a sealing membrane having a first surface and an opposite second surface, the sealing membrane configured to movably seal the array of microreactors. The microreactor array system further includes a reagent gap providing a fluid path between the array of microreactors and the second surface of the sealing membrane when the second surface of the sealing membrane is in spaced relationship with the array of microreactors. The microreactor array system further includes an injector for delivering a reagent into the reagent gap, an applicator for directing a working fluid against the first surface of the sealing membrane, and a detector.

The microreactor array system may include a system for creating a pressure differential between the reagent in the injector and the reagent gap. The sealing membrane of the microreactor array system may be flexible. The sealing membrane may be impervious to liquids, providing a separation between the reagent and the working fluid. The sealing membrane may be more hydrophobic than at least a portion of a surface defining the array of microreactors. The sealing membrane may comprise the array of microreactors.

In one version of the microreactor array system, at least one of the microreactors includes functionalizing chemicals dispersed thereon. The functionalizing chemicals may be immobilized temporarily. The functionalizing chemicals may be immobilized using a soluble coating.

In one version of the microreactor array system, at least a portion of the sealing membrane includes functionalizing chemicals dispersed on the second surface. The functionalizing chemicals may be immobilized temporarily. The functionalizing chemicals may be immobilized using a soluble coating.

The microreactor array system may comprise initiating functionalizing chemical spots and reacting functionalizing chemical spots arrayed separately onto the array of microreactors and opposing flat surface that comes into contact with the array of microreactors, the initiating functionalizing chemical spots and the reacting functionalizing chemical spots being aligned with each other thus enabling multiplexed combinatorial chemical reactions. The microreactor array system may comprise a window configured with a window inlet port and a window outlet port for flowing the working fluid. A space between the window and microreactor array may define a gap for at least one of the reagent, the sealing membrane, and the working fluid. A vacuum may be applied using the system via at least one of the window outlet port and window inlet port to maintain at least a portion of the reagent gap open.

The microreactor array system may comprise a base configured with a base inlet port and a base outlet port for injecting or removing the reagent. A vacuum may be applied to the reagent gap using the system via at least one of the base outlet port and base inlet port.

In one version of the microreactor array system, the reagent is injected into the array of microreactors via a pressure differential generated by the system. The working fluid may be injected against the first surface of the sealing membrane via a pressure differential generated by the system. The viscosity of the working fluid may be higher than the viscosity of the reagent. The working fluid may be incompressible.

In one version of the microreactor array system, the applicator comprises one of a pneumatic source, a hydraulic source or a mechanical spring source. The reagent injector and working fluid applicator may be actuated using automated control. The microreactor array system may undergo thermal cycling.

In one version of the microreactor array system, the detector is an optical detector. The detector may be a fluorescence detector. The detector may comprise a CCD camera. The detector may be a surface plasmon resonance detector.

In one version of the microreactor array system, the sealing membrane is optically clear. The window may be optically clear. The window may be glass. The working fluid may be optically clear. The sealing membrane, window or working fluid may have low autofluorescence.

According to another embodiment of the present disclosure, a microreactor array system includes a microreactor array, and a sealing membrane having a first surface and an opposite second surface, the sealing membrane configured to movably seal the array of microreactors. The microreactor array system further includes a reagent gap providing a fluid path between the microreactors array and the second surface of the sealing membrane when the second surface of the sealing membrane is in spaced relationship with the array of microreactors. The microreactor array system further includes an inlet for delivering a reagent into the reagent gap, a window disposed above the first surface of the sealing membrane, an inlet positioned in the window for directing a working fluid against the first surface of the sealing membrane, and a detector for detecting a characteristic of the microreactor array. The window may be optically clear. The sealing membrane may comprise an optically clear material. The working fluid may be optically clear. The sealing membrane, window or working fluid may have low autofluorescence.

According to a further embodiment of the present disclosure, a method for sealing a reagent in microreactors of an array of microreactors includes providing a microreactor array system, comprising an array of microreactors, a sealing membrane having a first surface and an opposite second surface, the sealing membrane configured to movably seal the array of microreactors, a reagent gap providing a fluid path between the array of microreactors and the second surface of the sealing membrane when the second surface of the sealing membrane is in spaced relationship with the array of microreactors, and an injector for delivering a reagent into the reagent gap. The method further includes injecting the reagent into the array of microreactors using the injector, directing, using an applicator, a working fluid against the first surface of the sealing membrane to achieve a contact of at least a portion of the second surface with the array of microreactors, and detecting a characteristic of the microreactor array.

In one version of the method, the microreactor array system may further comprise a system for creating a pressure differential between the reagent in the injector and the reagent gap; and the method may further comprise applying a vacuum to the reagent gap using the system via a pressure differential generated by the system.

In one version of the method, the sealing membrane is flexible. The sealing membrane may be impervious to liquids, providing a separation between the reagent and the working fluid. The sealing membrane may be more hydrophobic than at least a portion of a surface defining the array of microreactors. The sealing membrane may comprise the array of microreactors.

In one version of the method, at least one of the microreactors includes functionalizing chemicals dispersed thereon. The functionalizing chemicals may be immobilized temporarily. The functionalizing chemicals may be immobilized using a soluble coating.

In one version of the method, at least a portion of the sealing membrane includes functionalizing chemicals dispersed on the second surface. The functionalizing chemicals may be immobilized temporarily. The functionalizing chemicals may be immobilized using a soluble coating.

In one version of the method, the microreactor array system further comprises a plurality of initiating functionalizing chemical spots and a plurality of reacting functionalizing chemical spots arrayed separately onto the array of microreactors and the opposing flat surface that comes into contact with the array of microreactors, wherein the initiating functionalizing chemical spots and the reacting functionalizing chemical spots are aligned with each other thus enabling multiplexed combinatorial chemical reactions.

In one version of the method, the microreactor array system further comprises a window configured with a window inlet port and a window outlet port for flowing the working fluid. The microreactor array system may further comprise a space between the window and microreactor array defining a space for at least one of the reagent, the sealing membrane, and the working fluid. The method may further comprise applying a vacuum using the system via at least one of the window outlet port and the window inlet port to maintain at least a portion of the reagent gap open.

In one version of the method, the microreactor array system further comprises a base configured with a base inlet port and a base outlet port for at least one of injecting and removing the reagent. The method may further comprise applying a vacuum to the reagent gap using the system via at least one of the base outlet port and base inlet port.

In the method, directing the working fluid against the first surface of the sealing membrane may further comprise displacing the reagent from the microreactor array system via at least one of the base inlet port and the base outlet port.

In the method, the working fluid may be injected against the first surface of the sealing membrane via a pressure differential generated by the system. The viscosity of the working fluid may be higher than the viscosity of the reagent. The working fluid may be incompressible. In one version of the method, the applicator comprises one of a pneumatic source, a hydraulic source, and a mechanical spring source.

In one version of the method, the reagent injector and working fluid applicator are actuated using automated control. In the method, the microreactor array system may undergo thermal cycling.

In one version of the method, injecting the reagent into the array of microreactors using the injector may further comprise separating the second surface of the sealing membrane and the array of microreactors.

These and other features, aspects, and advantages of the present invention will become better understood upon consideration of the following detailed description, drawings, and appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration of an isometric view of an embodiment base of a microreactor array system according to the present disclosure.

FIG. 2 is a schematic illustration of an isometric view of an embodiment of a microreactor array system according to the present disclosure.

FIG. 3 is a schematic illustration of a top plan view of the microreactor array system of FIG. 2.

FIG. 4 is a is a schematic illustration of a top plan view similar to FIG. 3 of an embodiment of a microreactor array system with larger functionalizing-chemical spots spanning more than one smaller microreactor.

FIG. 5 is a schematic illustration of a cross-sectional view of the microreactor array system of FIG. 3 as taken along the line 5-5, and further illustrating filling of the microreactors with reagent, wherein the sealing membrane second surface is initially not touching the microreactor array surface.

FIG. 6 is a schematic illustration of a cross-sectional view similar to FIG. 5 illustrating filling of the microreactors with reagent, wherein the sealing membrane second surface is initially touching the microreactor array surface.

FIG. 7 is a schematic illustration of a cross-sectional view similar to FIG. 5 illustrating the displacement of reagent with working fluid and sealing of the microreactors.

FIG. 8 is a schematic illustration of a top plan view of another embodiment of a microreactor array system illustrating an array of chambers patterned on the sealing membrane.

FIG. 9 is a schematic illustration of a cross-sectional view of the microreactor array system of FIG. 8 as taken along the line 9-9.

FIG. 10 is a schematic illustration of an embodiment of a system including a detection subsystem, an automation subsystem, and a microreactor array system according to the present disclosure.

FIG. 11 is a schematic illustration of a top plan view of an embodiment of a microreactor array system according to the present disclosure.

FIG. 12 is a schematic illustration of a cross-sectional view as taken along the line 12-12 of FIG. 11 showing the process of displacing excess reagent and sealing microreactors.

FIG. 13 is a schematic illustration of a top plan view similar to FIG. 11 showing another embodiment of a microreactor array system.

FIG. 14 is a schematic illustration of a cross-sectional view as taken along the line 14-14 of FIG. 13 showing the process of displacing excess reagent and sealing microreactors.

FIG. 15 is an optical image rendered with proteins expressed from DNA in small chemical reaction chambers (i.e., microreactors). DNA was printed into the microreactors on a 1″×3″ microreactor array. Proteins were expressed in situ from the DNA using in vitro transcription translation (IVTT) reagent. Microreactors were sealed during expression to prevent diffusion. Varying concentrations of DNA were printed to produce varying intensities of fluorescently labeled protein spots. The image has about 12 kilopixel resolution (108×108 spots) and has a width of about 1.5 inches and a height of about 2 inches. Original photograph of Albert Einstein by Philippe Halsman @ Halsman Archive.

FIG. 16 is an example signal to background ratio bar chart of antibody response in patient serum sample detected by proteins expressed on a microreactor array vs. flat glass. Anti-p53 antibody signal to background ratio is plotted as a function of serum dilutions of 1:50, 1:100, 1:300, and 1:900 for both microreactor array and flat glass slide.

FIG. 17 is a schematic of an automated integrated microreactor array chemical processing system.

FIG. 18 is a schematic of an epifluorescent microreactor array optical detection system.

FIG. 19 is a schematic of oblique illumination microreactor array fluorescent optical detection system.

FIG. 20 is a schematic of a surface plasmon resonance (SPR) microreactor array optical detection system.

FIG. 21 is a schematic illustration of an example method for a microreactor array end-to-end biochemical assay processing protocol.

FIG. 22 is a fluorescent image of microreactors while they were inside of an integrated microreactor array system of the present disclosure.

### DETAILED DESCRIPTION

The present disclosure overcomes the drawbacks of previous technologies by providing a system and method directed to, in certain embodiments, an automated, high-throughput system with integrated detection to automate multiplexed assays.

The system may enable the execution of thousands of unique simultaneous biochemical reactions in an array of sealed microreactors. By sealing the microreactors, each microreactor is isolated from each other microreactor, thereby reducing or preventing interference between microreactors and with the external environment. Reactions may take place under identical conditions of temperature, sample concentration, sample composition and washing, blocking, and drying protocols. The system may provide orders of magnitude improvements over existing assays in terms of greater numbers of analytes, smaller sample volumes, smaller reagent volumes and higher sensitivity of assay. The system may include an array of microreactors accessed in parallel by a thin sheet of reagent and then sealed by complimentary working fluid acting through a flexible sealing membrane. Chemical reactions may be contained within the sealed microreactors. In one example, the system may provide an automated multi-step system for chemical reaction assays such as biochemical assays in the life sciences. Integrated optical detection may provide highly sensitive real-time detection based on enzyme-linked fluorogenic or chromogenic signal amplification or based on surface plasmon resonance.

In another aspect, the system may provide a laboratory automation instrument for the discovery of new biomarkers for early diagnosis of disease and insights into complex molecular pathways for new therapies. For example, the system may be used to monitor thousands of unique molecular interactions with a sample from a patient for comparison against clinical data. The system and method of the present disclosure may further provide for new clinical diagnostic systems.

In one embodiment, a system according to the present disclosure may include a microreactor array system or device for end-to-end, multi-step molecular detection assays. In one example, the system may be used to fill a microreactor array with cell-free in vitro transcription translation (IVTT) reagent, seal the wells, and incubate with temperature control. Thereafter, the system may be used to carry out various steps including washing, blocking, labeling with primary and secondary antibody, and detection using an integrated optical detector. The system may carry out the steps with manual or automatic control. Further, one or more steps may be carried out without disassembling the microreactor array system to recover the microreactor array.

As described previously, a nucleic acid programmable protein arrays (NAPPA) may be constructed by spotting protein-encoding plasmid DNA at high density, in addressable fashion, on a microreactor array surface. Subsequently, proteins may be generated in situ just prior to experimentation using cell-free IVTT expression systems. The NAPPA system may overcome challenges presented by spotted protein arrays, thereby enabling diverse functional protein studies, including the study of protein-small molecule, protein-protein, antigen-antibody, and protein-nucleic acid interactions. The present system may further offer a versatile and adaptable system amenable to a variety of capture modalities and expression systems. Further, the system may, in some embodiments, be implemented with a microarray printer and laser slide scanner. Embodiments of the present system may facilitate NAPPA-ELISA by integrating real time optical detection with a microreactor array system. Further embodiments of a system may include automated end-to-end processing methodology using computer controlled valves and pumps.

In one aspect, the present disclosure provides an automated high-throughput system for multiplexed assays in the life sciences. The instrument may be used for research to study biomolecular interactions correlated to clinical data, adapted for laboratory diagnostics, the like, and combinations thereof. The present disclosure may transform high-throughput experiments in the life sciences by increasing the number of simultaneous multiplexed biochemical reaction assays by two orders of magnitude from 96-well or 384-well microplates to over 20,000 per microreactor array. Further, the present disclosure may provide a small-scale microplate assay system with dimensions on the order of a microscope slide. In some embodiments, the present disclosure may provide a microreactor array system that uses a total reagent volume of about 300 μl or less. In one aspect, the present disclosure may enable complex molecular screening assays involving tens of thousands of molecular targets and hundreds of clinical patient samples. High-throughput assays may help elucidate connections between molecular interactions and disease. High sensitivity, due to enzymatically linked fluorescent substrate signal amplification, may enable improved limit of detection (LOD) of rare, low abundance biomarkers.

In some embodiments, a system may include both high speed non-contact piezoelectric dispensing and microreactor array technology. The microreactor array system may enable multiple, independent, chemical reactions in a parallel, high-throughput format. Individual chemical reactions are physically isolated in sealed containers thus preventing evaporation or diffusion during an experiment. Each container, ‘microreactor’, may contain unique, untethered, free floating intermediate molecular compounds that undergo isolated chemical reactions without interference from neighboring reactions or external environments. All of the reactions may take place simultaneously on a single microscope slide format microreactor array under identical operating conditions.

A system according to the present disclosure may enable filling tens of thousands of microreactors per second with reagent and then rapidly sealing all of them using viscous, pressurized working fluid acting through a flexible sealing membrane. In one aspect, the system was validated with NAPPA that express thousands of functional proteins from genomic DNA printed in a microarray format. Densities greater than 10,000 expressed protein spots were demonstrated with no diffusion of free floating molecules during a three hour period of incubation. Using the device to probe for an autoantibody cancer biomarker in blood serum sample gave five-fold greater normalized detection signal compared with a standard protein microarray expressed on a flat microscope slide.

In one example, a microreactor array includes about 14,000 microreactors in a hexagonal array pattern etched into a 25.4 mm×76.2 mm silicon wafer. Individual microreactors may be filled with different unique functionalizing chemicals using non-contact piezoelectric inkjet dispensing technology. Portions of these chemicals may be bound to the functionalized surfaces of the microreactors. The slides may be soaked in a blocking buffer to wash away remaining unbound chemicals or to mitigate nonspecific binding. After rinsing and drying, slides may be inserted into the device. A sealing gasket or O-ring may be placed around the microreactor array for vacuum or pressure sealing. A transparent, flexible, impenetrable, smooth, sealing membrane may be placed over the sealing gasket or O-ring and slide. A transparent window may be placed over the sealing membrane and the assembly may be clamped together in a rigid frame using fasteners.

Microreactors are filled with reagent at a rate of about 10,000 microreactors per second. The functionalizing chemicals may remain in place during filling as they are bound to the surface. High viscosity, pressurized, incompressible, working fluid may be injected onto the sealing membrane through an inlet port in the window. The sealing membrane may wipe excess reagent from the slide surface as the working fluid spreads out across the membrane. After spreading out across the whole surface, the pressurized working fluid may continue to apply even pressure onto the microreactor array surface through the sealing membrane. In one aspect, this may uniformly seal all of the microreactors, thereby isolating the microreactors from one another.

The process of filling and sealing all of the microreactors may take less than about five seconds. The process may be actuated entirely by opening or closing valves either manually, through the use of an automated control system, or a combination thereof. Reagent may react with functionalizing chemicals in the microreactors to create untethered intermediate molecules that are contained for extended incubation periods. After incubation, the slide may be removed from the device for further chemical processing such as washing, blocking or labeling. To preserve molecular functionality, the microreactor array may be prevented from drying out during an assay. The whole microreactor array (or a portion thereof) may be flooded with sample fluid to detect probe molecules by their affinity to specific chemical products or targets in the microreactors. After chemical processing, the microreactor array may be dried and imaged using a fluorescent microarray scanner or another detection system. The detection system may be integrated into the microreactor array system.

### 1. Microreactor Array System

The present disclosure provides a microreactor array system that enables simultaneous, multiple, parallel, high-density, independent, unique chemical reactions involving possibly free floating molecular compounds. Individual chemical reactions may thus be physically isolated in sealed containers, cavities, or chambers, preventing evaporation, diffusion or movement during an experiment. Chemical reactions in neighboring sealed containers, cavities, or chambers, therefore may not interfere with each other. As such, each sealed container, cavity, or chamber may contain unique molecular compounds that may undergo isolated chemical reactions without interference from neighboring containers, cavities, or chambers, or other external environments. In this manner, the microreactor array system allows for any number of parallel reactions to take place at the same time, and under identical chemical and operating conditions, such as, for example concentration, volume, temperature, pressure and so on. Physical principles for design of the microreactor array system are presented in the following paragraphs. These are followed by operational design principles for filling microreactors with reagent, displacing excess reagent and sealing the microreactors.

Good performance of a microreactor array system may be characterized by complete filling and sealing of the microreactors. It is desirable that the microreactors be completely filled with reagent without entrapped air pockets and be sealed completely to prevent leakage for the duration of an experiment. Parameters of the microreactor array system are very interdependent. For example, working fluid pressure required to effectively seal the microreactors may depend on sealing membrane thickness.

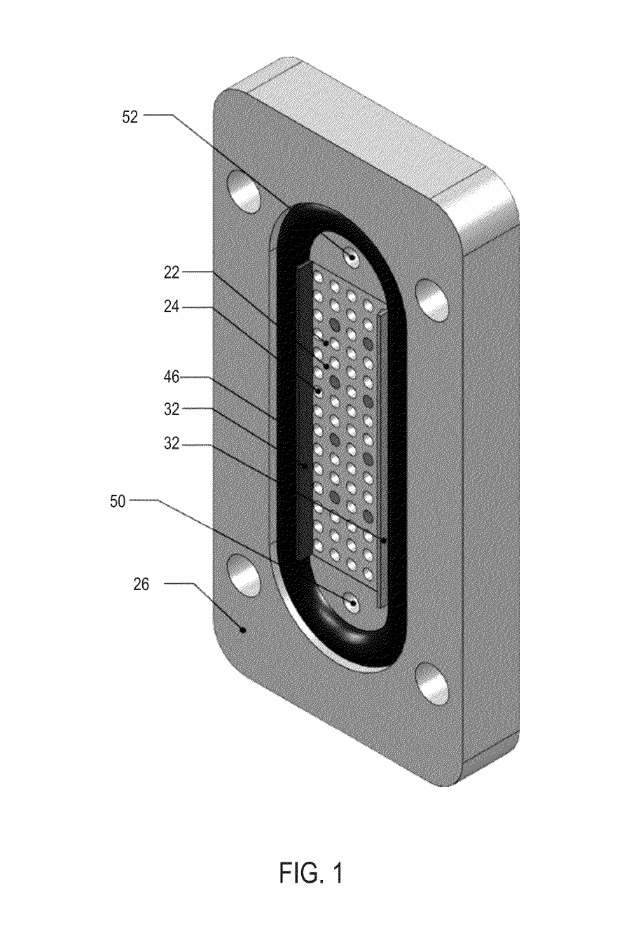

Turning now to the Figures, an embodiment of a microreactor array includes a microreactor array 22 including a plurality of microreactors 24. The microreactor array 22 may be reversibly insertable into a housing or base 26. The base 26 may have a plate or slab-like configuration including a central cavity 26a for receiving the microreactor array 22 therein. In the illustrated embodiments, the microreactor array system 20 is configured for filling microreactors 24 with one or more materials (e.g., fluids, buffers, samples, reagents, etc.) and sealing the microreactors 24 with a sealing membrane 28. Referring at least to FIG. 1, the microreactor array 22 may be fashioned from a single layer, which may be a flat rigid surface constructed using any appropriate material. The single layer may include an array of cavities, chambers, or microreactors 24 of any desired shape, size and depth, patterned into the upper surface 22a of the microreactor array 22. In some configurations, the microreactors 24 may also be constructed from a number of separate layers. For example, the microreactor array 22 may be formed from a first layer (e.g., a glass microscope slide) and a second layer arranged adjacent to the first layer, the second layer including an array of through holes of any desired shape and size.

In general, microreactor arrays may be manufactured from silicon wafers using standard silicon wafer fabrication processes from the semiconductor industry. Silicon wafers are readily available, relatively inexpensive and have precise thickness tolerance and consistent material properties. Silicon surfaces can be oxidized with a thin silicon dioxide (SiO₂) layer to be compatible with conventional glass surface chemistry and to prevent fluorescent signal quenching of bare silicon. Microreactor arrays manufactured from silicon may be reused by stripping off the SiO₂ layer and re-oxidizing the surface. The SiO₂ layer may be selectively patterned on the surface to provide areas of fluorescent quenching vs. non-quenching. Similarly, the surface of the microreactor array may be patterned to be more hydrophobic than the inside of the microreactors. Silicon has good thermal conductivity which may assist speed and uniformity of thermal cycling. Microreactor arrays may also be manufactured from other materials such as glass, polydimethylsiloxane (PDMS) and so on.

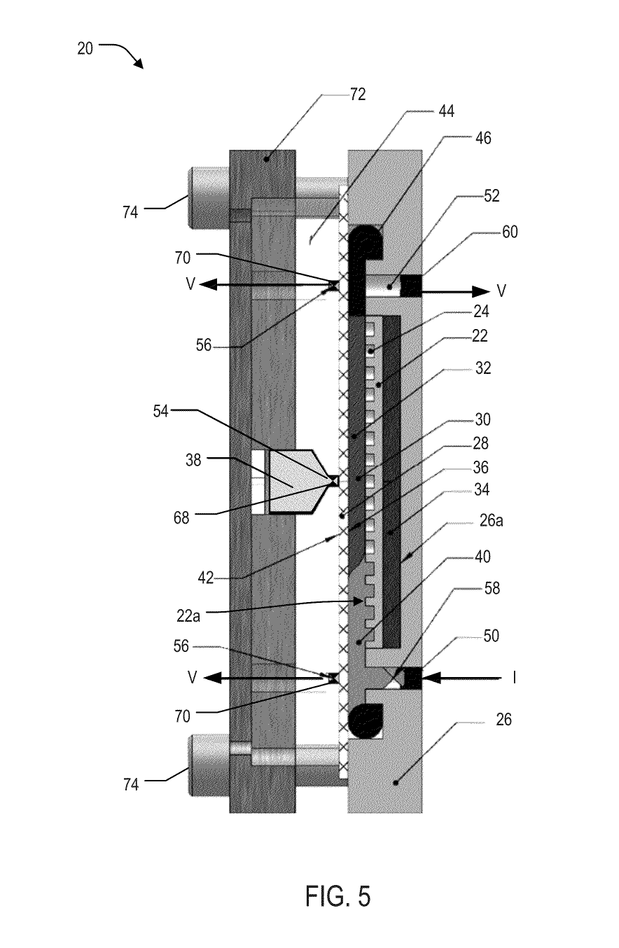

Microreactor arrays that are the same dimensions as microscope slides, for example, 25 mm×75 mm×1 mm (˜1″×˜3″× ˜0.04″), may be used in standard microarray scanners. Depending on the type of microarray scanner, bare reflective areas may have to be provided on the two ends of microreactor array surface to enable autofocus. Focus depth may be adjusted for the depth of the microreactors. In one aspect, it may be useful to manufacture the microreactor array 22 to precise mechanical tolerances so that the microreactor array 22 fits into the base 26 of the microreactor array system 20. Further, when positioned within the base 26, the microreactor array 22 may define a reagent gap 30 having a precise thickness as shown at least in FIG. 5. Alternatively (or additionally), one or more shims 32 positioned to extend outwards from an upper surface 22a of the microreactor array 22, a compliant layer 34 positioned beneath the microreactor array 22, or a combination thereof may be provided to define a precise reagent gap 30. Accordingly, the base 26 may be sized to accommodate one or more of the shims 32 adjacent the sides 22b of the microreactor array 22 in the cavity 26a of the base 26. Further, the base 26 may be sized to accommodate a compliant layer 34 beneath at least a portion of the microreactor array 22.

Microreactors 24 in a microreactor array 22 may be distributed in any desired pattern and may include any number of microreactors 24. For example, a hexagonal pattern may fit more circular microreactors for the same pitch and area compared to rectangular patterns. Each microreactor 24 may be identified by a row and or column location in the microreactor array 22. Also, hexagonal patterns may help during filling of the microreactors 24 by breaking up the flow of reagent as opposed to rectangular patterns that may allow flow of reagent along straight fluid paths between columns of microreactors 24.

The size, shape and depth of the microreactors 24 may affect functionalizing, filling, sealing and results of chemical reactions. Wider microreactors may be easier to target during an initial filling with functionalizing chemicals, for example, using piezoelectric inkjet dispensing. Also, wider microreactors may have thinner borders which may help with filling with reagent, since there is smaller surface area to channel flow around each microreactor. Thinner borders also may help with sealing by increasing sealing pressure for the same applied force. For example, in a hexagonal array pattern, hexagonal microreactors may help with functionalizing, filling and sealing, since they have consistent border thicknesses compared to circular microreactors. In addition, depth to width ratio (aspect ratio) of the microreactors may also be important for filling. For example, an aspect ratio less than 1, corresponding to a shallow microreactor, may be advantageous in filling microreactors without entrapping air pockets. In particular, this may be applicable when initially filling individual microreactors with any functionalizing chemicals and subsequently filling them with reagent. Bigger microreactors have proportionally greater volume to surface-area than smaller ones. This may favor chemical reactions that are limited by the volume of the reagent. Conversely, smaller microreactors may favor chemical reactions that are limited by the amount of chemicals functionalized on their surfaces. Therefore, size factors may need to be accounted for when conventional chemical reactions are scaled down to microreactors. Relative concentrations of functionalizing chemicals and reagent may have to be adjusted accordingly.

In addition, microreactors with rounded corners may be easier to fill than microreactors with sharp or angular corners. This may be applicable for initial filling of microreactors with functionalizing chemicals, and subsequently with reagent. In particular, functionalizing chemicals tend to be pulled into sharp corners, resulting in non-uniform coating of microreactor surfaces. This may end up resulting in non uniform fluorescent signal intensity across the surface of the microreactors. Furthermore, this may also result in non-uniform wetting across the microreactors which may entrap air pockets when subsequently filling microreactors with reagent. Microreactors with uniformly hydrophilic (high wettability) surfaces may be easier to fill with functionalizing chemicals or reagent. Microreactor arrays with smooth top surfaces may also be easier to fill, with reagent, than ones with features that may obstruct the flow of reagent or direct it away from the microreactors.

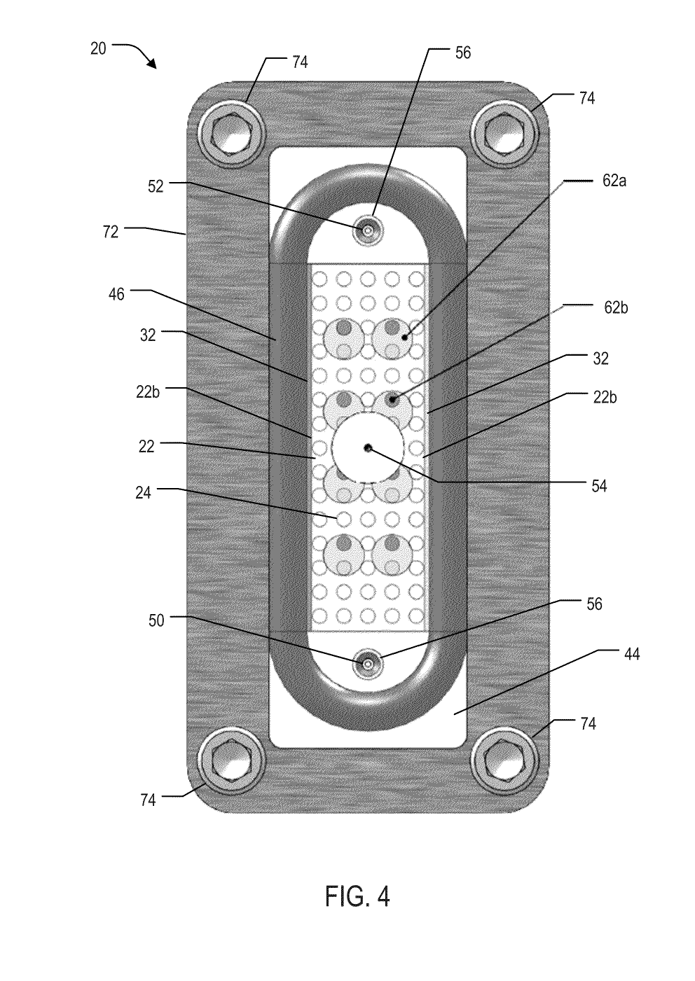

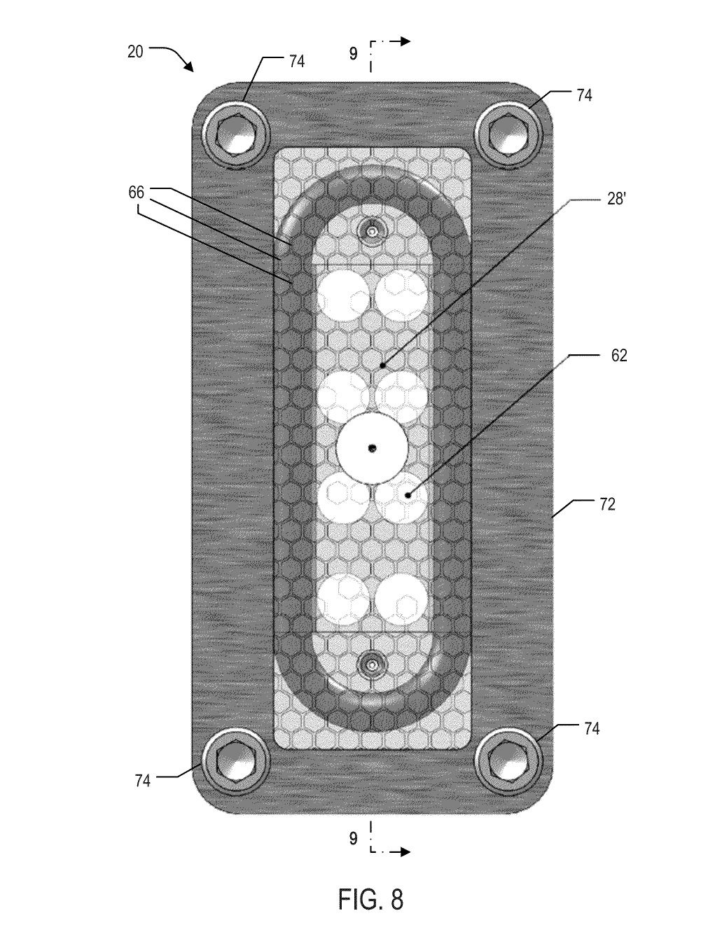

As described, microreactors 24 in the microreactor array 22 may be functionalized with any number of functionalizing chemicals either prior to usage or during operation. These functionalizing chemicals may be distinctive with respect to any number of microreactors 24 and may be dispensed or dispersed along the upper surface 22a of the microreactor array 22 by any number of loading processes, such as pin spotting, piezoelectric inkjet dispensing or chemical synthesis process. Functionalizing chemicals may either initiate chemical reactions or react with products of chemical reactions. Initiating functionalizing chemicals may include any substances, such as PCR primers, that initiate chemical reactions to produce unique products, such as specific amplified DNA. Reacting functionalizing chemicals may include molecular targets that bind specific probe molecules in a sample or bind the products of chemical reactions in microreactors 24. The upper surface 22a of the microreactor array 22 and the opposing flat surface (e.g., the second surface 36 of the sealing membrane 28) that it comes into contact may each be separately functionalized with chemical spots 62 to initiate chemical reactions or react with products of those reactions respectively. The initiating chemical spots 62a and reacting chemical spots 62b may either be coated onto the entire sealing membrane 28 or on any surface of the microreactor array 22 (e.g., upper surface 22a, the side walls or bases of the microreactors 24, or the like) or arranged in an array of chemical spots 62. Initiating chemical spots 62a and reacting chemical spots 62b may both be arrayed separately onto the microreactor array 22 or the opposing flat surface (e.g., sealing membrane 28) and then aligned with each other thus enabling multiplexed combinatorial chemical reaction experiments. For example, the initiating chemical spots 62a may be dispensed about or embedded in the microreactors 24 prior to use. In this case, the opposing flat surface that comes into contact with the upper surface 22a of the microreactor array 22 may be functionalized separately to react with products of chemical reactions taking place in microreactors 24. Alternatively (or in addition), initiating chemical spots 62b may be dispensed or dispersed onto the opposing flat surface that comes into contact with the upper surface 22a of the microreactor array 22. In this case, any portion of the microreactor array 22 may be functionalized separately to react with products of chemical reactions taking place in microreactors 24. In some aspects, individual larger functionalizing chemical spots 62 may span multiple smaller microreactors 24 as shown at least in FIGS. 4 and 8, so that precise alignment of functionalizing chemical spots 62 with microreactors 24 may not be required when dispensing functionalizing chemicals into microreactors 24. Likewise, if functionalizing chemical spots 62 are arranged onto an opposing flat surface, precise alignment of the microreactor array 22 and opposing flat surface may not be required if larger functionalizing chemical spots 62 span multiple smaller microreactors 24.

The microreactor array system 20 also includes a flexible sealing membrane 28 which is configured to provide a movable seal or cover for the array 22 of microreactors 24 by coming into contact with the microreactor array 22. In some embodiments, the microreactor array 22 may be formed using an opposing flat surface 64 and an array of containers, cavities, or chambers 66 patterned on the sealing membrane 28. In one aspect, the opposing flat surface may be a glass microscope slide, a silicon wafer, or another suitable plastic, glass, metal, composite material, or like substrate.

In some embodiments, the sealing membrane 28 represents an important component of the microreactor array system 20. During operation, the sealing membrane 28 may be placed in direct contact with contents of the microreactors 24. Further, the sealing membrane 28 may be designed to not interfere with chemical reactions taking place within the microreactors 24. Since the sealing membrane 28 may separate a working fluid 38 from a reagent 40, the sealing membrane 28 may be impenetrable to fluids, without holes or permeability to fluids. In one aspect, it may be useful to provide a sealing membrane 28 that is strong enough to withstand high sealing pressures yet be thin and compliant to conform to and seal the microreactors 24. Lower sealing pressures may be used with thinner sealing membranes 28. Note that arrows designated with a “P” in the Figures indicate the application of pressure. The sealing membrane 28 may deform smoothly into the reagent gap 30 when sealing the microreactors 24. The sealing membrane 28 may be inert to the chemicals with which it comes into contact. Also, the sealing membrane 28 may withstand temperature variations due to thermal cycling or exothermic chemical reactions, endothermic chemical reactions, or a combination thereof. In some embodiments, the sealing membrane 28 may be stiff laterally so that the sealing membrane 28 lies flat, without wrinkles, prior to filling with reagent 40. In other words, the sealing membrane 28 may be made from a rigid material like polyester instead of softer material like low density polyethylene (LDPE) or fluorinated ethylene propylene (FEP). A first surface 42 and the second surface 36 of the sealing membrane 28 may provide smooth, uniform flow paths for working fluid 38 and reagent 40 respectively. The sealing membrane 28 may be free of nicks, creases, wrinkles, gashes, holes, dust, dirt or other defects that may trap reagent 40 or otherwise cause improper localized sealing of the microreactors 24. The second surface 36 of the sealing membrane 28 may be uniformly less wettable (more hydrophobic) than the surfaces of the microreactor array 22 (more hydrophilic) to help fill the microreactors 24 with reagent 40. The sealing membrane 28 may be transparent so that the surfaces of the microreactor array 22 can be seen through a window 44 and sealing membrane 28.

All of the functionalizing chemicals, reagents or other materials in each of the microreactors 24 may interact with a single reagent 40, which may, for example, be a reagent that initiates chemical reactions or a sample containing different target molecules to be identified. The microreactor array system 20 enables rapidly filling of the microreactors 24 with reagent 40 and then sealing them into independent chemical reaction containers (i.e., microreactors 24).

The microreactor array system 20 may include a sealing gasket or O-ring 46 to pneumatically or hydraulically seal the microreactor array 22 around its periphery. A transparent window 44 may allow observation of any filling processes. The bottom surface of the window 44 may provide a smooth consistent flow path for reagent 40 and working fluid 38. An inlet port 50 and an outlet port 52 may provide for injecting a reagent 40. Further, an inlet port 54 and an outlet port 56 may provide for injecting a working fluid 38. Inside diameters of the liquid paths of the ports (i.e., inlet port 50, outlet port 52, inlet port 54, and outlet port 56) may be larger than the reagent gap 30 so that liquid flow is not restricted by the ports. Liquid paths of the ports may be short to reduce dead-volume and flow resistance.

Microreactors 24 may be filled by forcing reagent 40 through a thin reagent gap 30 between the second surface 36 of the sealing membrane 28 and the microreactor array 22. For consistent, reliable filling of the microreactors 24, with minimal reagent volume, the thickness of the reagent gap 30 may be controlled. For example, the thickness of the reagent gap 30 may be controlled by tight dimensional tolerances of the microreactor array 22 and microreactor array system 20. Likewise, the microreactor array system 20 may be mechanically stiff to maintain a precise reagent gap 30 even under the stress of clamping the microreactor array system 20 together. Mechanical stiffness and strength may also be required to withstand the stresses of a working fluid pressure and to prevent leaks. Stiffness of the microreactor array system 20 may be achieved by a combination of stiff materials and thick base 26, top 72 and window 44. A precise reagent gap may also be achieved using shims 32 on the upper surface 22a of the microreactor array 22 along the edges as shown in FIG. 1. In this case, a compliant layer 34 may be placed underneath the microreactor array 22, and compressed when the microreactor array system 20 is clamped together.

Experiments in the life sciences are typically carried out in aqueous solutions. Microreactors may be filled with aqueous reagents to initiate chemical reactions for experiments in the life sciences. Water has high surface tension which dominates other forces at small dimensions. For example, high surface tension causes air to become trapped in microreactors 24 if the microreactor array 22 is submerged in water. The same thing happens if water is poured on top of the microreactor array 22. Surface tension tends to keeps liquid surfaces smooth with minimal surface area. However, the surface of a reagent must be deformed into thousands of tiny bumps to conform to the indentations of microreactors to fill them with reagent. Reagent 40 may be forced into microreactors 24 by applying vacuum to a thin reagent gap 30 and then quickly injecting reagent 40, under pressure, into the reagent gap 30. This approach overcomes surface tension through a combination of inertial, pressure, vacuum, wetting and viscous forces to force reagent into tiny microreactors 24. Note that arrows designated with a “V” in the Figures indicate the application of vacuum, while arrows designated with an “I” in the Figures indicate a point of injection. Reagent 40 may be injected into the inlet port 50 with a manual or automated syringe or pipette, imparting kinetic and potential energy to overcome the surface energy of the fluid. In some cases atmospheric pressure may be sufficient to reliably fill microreactors 24 with reagent 40. In this case, reagent 40 may be injected into the inlet port 50 through a solenoid valve or another type of valve. In general, a ‘stronger’ fluid in terms of pressure, density, incompressibility and viscosity pushes ‘weaker’ fluid out of its way. During filling, air remaining in microreactors 24 is displaced by an advancing wall of pressurized reagent 40. Similarly, after filling, excess reagent 40 is displaced from the upper surface 22a of the microreactor array 22 prior to sealing. This is done using an advancing wall of pressurized high viscosity working fluid 38 that displaces excess reagent 40 through the sealing membrane 28. Similar issues, concerning filling small features with fluids, come up in other fields such as plastic injection molding and microimprint lithography. Note that arrows designated with a “D” in the figures indicate the displacement of a fluid or other material.

The fluid mechanics dimensionless parameters that govern filling of microreactors 24 with reagent 40 are outlined in the following seven paragraphs.

Body (gravitational) forces may have little influence on filling microreactors 24. Bond number

$$Bo = \frac{ρ {a ( {d}_{h} )}^{2}}{γ}$$

characterizes the relative influence of body force ρα(dₕ)² vs. surface tension Y. Body force depends on density ρ, acceleration α, and hydraulic diameter dₕ. For thin closed channels, dₕ is twice the thickness of the channel d. If Bo<1, then surface tension dominates body forces. A microreactor array system 20 has low Bond number meaning that flow into microreactors 24 is dominated by surface tension vs. body forces. To fill microreactors 24 with liquid using body forces may require higher accelerations which may be achieved in a centrifuge. Microreactors 24 may also be filled by submerging them in liquid and degassing in a vacuum chamber. However, both of these approaches take time and chemical reactions may be well underway before the microreactor array 22 can be removed from the centrifuge or vacuum chamber and then sealed. Filling microreactors 24 by degassing in a vacuum chamber may require >5 minutes to pull air out of the microreactors 24 through the gas permeable layer of liquid on the upper surface 22a of the microreactor array 22. This process may be expedited by degassing the liquid prior to submerging a microreactor array or by heat or sonication while applying vacuum.

Air may need to be advantageously displaced from the microreactors 24 to fill them completely with reagent 40. The advancing front of reagent 40 pushes air out of the microreactors 24 and out through the outlet port 52. Air pressure increases towards the outlet port 52 of the microreactor array 22 possibly resulting in incomplete filling of those microreactors 24. Applying a vacuum at the outlet port 52, prior to filling, helps mitigate this problem. The goal is to remove as much air from the microreactors 24 as possible prior to injecting reagent 40. The air mass mᵥ in the microreactors 24 under vacuum P_{ν} compared to the mass mₐ at ambient pressure P_{α} is

$$\frac{{m}_{v}}{{m}_{a}} = \frac{{P}_{v}}{{P}_{a}} .$$

Therefore 97% of the air mass is removed from the microreactors 24 by applying a vacuum of about 29 inches Hg (about 0.92 inches Hg absolute pressure). The advancing fill front of the reagent 40 displaces the remaining air in the microreactors 24. The difference between the positive pressure of the reagent 40 and the vacuum pressure in the microreactors 24 helps force reagent 40 into the microreactors 24. The inlet port 50 for the reagent 40 may have a check valve 58 with cracking pressure greater than about 1 atmosphere to prevent reagent 40 from leaking into the reagent gap 30 when vacuum is applied. In some embodiments a burst valve may be substituted for or combined with the check valve 58 at the inlet port 50 to maintain vacuum before injecting the reagent 40. Pressure at the inlet port 50 may break the burst valve allowing reagent 40 to flow freely into the reagent gap 30 with minimal resistance. A hydrophobic restriction valve 60 at the outlet port 52 may prevent vacuum from sucking reagent 40 from the reagent gap 30 after filling the microreactors 24. In some embodiments, a check valve may be combined with or substituted for the hydrophobic restriction valve 60 at the outlet port 52 to maintain a vacuum in the reagent gap 30 before injecting reagent 40. In another aspect, the inlet port 50 and outlet port 52 may have actuated 3-way valves synchronized to switch from vacuum to reagent 40 during injection.

Reagent 40 is exposed to a large surface area when it is injected into the reagent gap 30. If vacuum is applied to the reagent gap 30 then gas dissolved in the reagent 40 may come out of solution when exposed to this vacuum. Therefore the reagent 40 may have to be degassed prior to injection to prevent gas bubbles forming in the reagent 40 during injection.

Weber number

$${W}_{e} = \frac{ρ {d}_{h} {v}^{2}}{γ}$$

characterizes relative influence of inertia vs. surface tension γ of fluid with density ρ flowing at a velocity ν in a film of thickness dₕ. It is used to analyze thin film flows and the formation of droplets and bubbles. The Weber number for a microreactor array system is Wₑ>1 meaning that inertial forces have moderate influence on filling microreactors compared to surface tension. Weber number scales as the square of velocity ν so it can be increased by injecting reagent 40 faster. However there is an upper limit to velocity for filling microreactors. At higher Weber numbers, reagent 40 may break up into droplets on the upper surface 22a of the microreactor array 22 due to inertial forces. This is exacerbated if gas is trapped or dissolved in the reagent 40.

Capillary number

$${C}_{a} = \frac{μ v}{γ}$$

characterizes relative influence of viscous μν vs. surface tension forces. The microreactor array system 20 may have low capillary number, meaning that liquid flow dynamics are heavily influenced by surface tension compared to viscosity μ. One group investigated liquid filling dynamics of small features at low Capillary numbers, Cₐ=0.001 to 0.01 for microimprint lithography applications. They identified the following conditions for complete filling of small features: high Capillary number, low aspect ratio features and high surface wetting properties. At high Capillary numbers, the dynamic contact angle becomes very largely dependent on global hydrodynamics of the flow possibly enabling high speed filling of microreactors. High Capillary number is achieved by high flow velocity, high viscosity or low surface tension. High flow velocity may come from an external pressure differential applied to the reagent 40. Relying on capillary action alone, without applying pressure, may result in incomplete filling of microreactors 24 since reagent 40 may flow slowly over the top of the upper surface 22a of the microreactor array 22 without going into the microreactors 24. Under certain circumstances, it may be possible to increase Capillary number by adding a thickening agent to the reagent 40 to increase viscosity. Similarly, Capillary number may be increased by decreasing surface tension with surfactant. Small surfactant concentrations may decrease surface tension dramatically. Aspect ratio is relative depth to width ratio of microreactors 24. Generally, aspect ratios less than 1 are required for complete filling of microreactors 24. In other words, it may be useful to provide microreactors 24 that are wider than they are deep for complete filling. Surface wetting properties in the reagent gap 30 may also be important. A higher wetting (hydrophilic) surface of microreactor array 22 compared to the opposing surface (hydrophobic) may promote filling of the microreactors 24. Hydrophilic surfaces have lower contact angle compared to hydrophobic ones which pulls liquid into the microreactors 24. Consistent wetting properties across the surface of the microreactors 24 may also prevent entrapping localized air pockets. Uniformly pre-coating the surfaces of the microreactor array 22 with a hydrophilic reagent may promote uniform wetting and filling of microreactors 24.

Reynolds number

$${R}_{e} = \frac{ρ {vd}_{h}}{µ}$$

characterizes relative influence of inertial vs. viscous forces. Physical design parameters are interdependent. In one aspect, it may be useful to balance physical design parameters to achieve proper filling of the microreactors. High reagent injection velocity increases Capillary number to help overcome surface tension with viscous forces. However, Weber number also increases with velocity, which may result in incomplete filling of microreactors due to inertial forces that may break up reagent flow. This may manifest as foaming and may be exacerbated by gas dissolved in the reagent. Inertia may also cause reagent to skim across the top of the microreactors instead of flowing down into them. Complete filling of microreactors may be assisted by a relatively low Weber number and a relatively high Capillary number. The ratio of Weber to Capillary numbers is also Reynolds number

$$Re = \frac{{w}_{e}}{{c}_{a}} .$$

For complete filling of microreactors, it may be useful to provide a low Reynolds number. For example, regent flow in the reagent gap of the microreactor array system may have a Reynolds number of about 20, thereby generally resulting in laminar flow. In one aspect, a low Reynolds number corresponds to low reagent injection velocity ν, a high kinematic viscosity μ/ρ, and a small reagent gap dₕ.

After filling the microreactors 24, excess reagent 40 is displaced from the upper surface 22a of the microreactor array 22 and each of the individual microreactors 24 is sealed to prevent evaporation, leakage, diffusion, movement or cross-contamination during an assay. Functionalizing chemicals may be immobilized to prevent dislodging by reagent 40 prior to sealing the microreactors 24. For example, functionalizing chemicals may be permanently immobilized by chemical bonds. Alternatively (or additionally), functionalizing chemicals may be immobilized only temporarily, for at least as long as it takes to fill the microreactors 24 with reagent 40 and then seal them. After the microreactors 24 are filled with reagent 40 and sealed, the functionalizing chemicals may be released into the reagent 40 without diffusing into neighboring microreactors 24. Temporary immobilization may be achieved via reversible chemical bonds, photocleavable linkers, magnetic beads, soluble coatings, or the like. A soluble coating may be applied, for example, via non-contact dispensing or aerosol spray. In some configurations, the soluble coating may be a polypeptide such as gelatin, bovine serum albumen (BSA), or the like. If functionalizing chemicals are arrayed onto a flat flexible sealing membrane 28, then they may be temporarily immobilized by overlaying them with a thin sheet of soluble material.

Excess reagent 40 may be displaced from the upper surface 22a of the microreactor array 22 after filling the microreactors 24 to ensure proper sealing. Trapped puddles of reagent 40 may impede complete sealing, thereby resulting in leakage, diffusion or cross-contamination of the contents of the microreactors 24. Therefore, it may be useful to configure the microreactor array system 20 for displacing excess reagent 40. Some type of wiping, rolling or squeegee action may be implemented to displace excess reagent 40 from the upper surface 22a of the microreactor array 22. Pushing straight down on the microreactor array 22 with a compliant material may not displace the entire reagent 40 since puddles may become trapped on the upper surface 22a of the microreactor array 22. The puddles may not be displaced regardless of how hard the compliant material is pushed down. This is because the areas surrounding the puddles may be sealed more tightly as the compliant material is pushed down, blocking the flow of reagent 40 away from the puddles. The incompressible liquid of a puddle may be pushed further into the compliant material without being displaced from the upper surface 22a of the microreactor array 22. A rigid material may displace more reagent 40. However, some liquid may be invariably trapped in valleys, caused by surface irregularities between the rigid material and the upper surface 22a of the microreactor array 22, resulting in poor sealing of microreactors 24 in those areas.

In some embodiments, the microreactor array system 20 displaces excess reagent 40 by forcing pressurized working fluid 38, through the inlet port 54 in the bottom of the window 44, against the first surface 42 of a sealing membrane 28. As the pressurized working fluid 38 spreads out against the first surface 42 of the sealing membrane 28, the working fluid 38 may displace reagent 40, from the upper surface 22a of the microreactor array 22, through contact with the second surface 36 of the sealing membrane 28. Excess reagent 40 is then pushed out through the inlet port 50 or outlet port 52 for the reagent. If desired, reagent 40 may then be recovered from the inlet port 50 or outlet port 52. The sealing membrane 28 may be impervious to liquids and may be smooth without any wrinkles or creases, which may trap reagent 40. In some embodiments, the working fluid 38 has a high viscosity to slowly wipe the reagent 40 away from the upper surface 22a of the microreactor array 22. The temperature of the working fluid 38 and the whole microreactor array system 20 may be lowered to increase the viscosity of the working fluid 38 prior to injection against the first surface 42 of the sealing membrane 28. A constant sealing pressure force may extrude the high viscosity working fluid 38 and may maintain a constant sealing pressure on the upper surface 22a of the microreactor array 22. Velocity of the working fluid is related to pressure via the Darcy-Weisbach equation

$$∇ p = {f}_{D} \frac{1}{{d}_{h}} \frac{ρ {v}^{2}}{2}$$

relating pressure drop ∇p to fluid velocity v and fluid density ρ in a channel of length l and hydraulic diameter dₕ. Parameter f_{D} is the dimensionless Darcy friction factor which is

$${f}_{D} = \frac{64}{{R}_{e}}$$

for laminar flow where Rₑ is the Reynolds number. The flow path for working fluid 38 is preferably smooth for uniform velocity against the sealing membrane 28. The viscosity of the working fluid 38 is preferably higher than the viscosity of the reagent 40 being displaced. If gas or low viscosity working fluid 38 is used, then working fluid 38 velocity may be too high to displace reagent 40 from the upper surface 22a of the microreactor array 22. Low viscosity working fluid 38 or gas may quickly spread out against the first surface 42 of the sealing membrane 28 without wiping the reagent 40 completely away from the upper surface 22a of the microreactor array 22. Consequently a thin layer of reagent 40 may become trapped on the upper surface 22a of the microreactor array 22 and may not be displaced regardless of how high a sealing pressure is applied to the working fluid 38. This trapped layer may prevent complete sealing of the microreactors 24 resulting in localized leakage of the contents of the microreactors 24. The outlet ports 56 of the window 44 may be kept open as the working fluid 38 spreads against the first surface 42 of the sealing membrane 28 to vent gas and prevent gas pressure buildup against the first surface 42 of the sealing membrane 28, thereby prematurely sealing the microreactors 24 before the working fluid 38 has a chance to displace the reagent 40 away from the upper surface 22a of the microreactor array 22. The outlet ports 56 of the window 44 may be closed once the working fluid 38 spreads out against the first surface 42 of the sealing membrane 28 to apply and maintain a constant pressure of the working fluid 38 through the sealing membrane 28 onto the upper surface 22a of the microreactor array 22 for the duration of the chemical reaction assay.

The sealing membrane 28 may temporarily adhere to the bottom surface of the window 44 without contact with the upper surface 22a of the microreactor array 22, if vacuum is applied to the reagent gap 30. In this case, the sealing membrane 28 may be adhered with a liquid adhesive that does not dry out. The sealing membrane 28 may then release smoothly when working fluid 38 is injected against first surface 42 of the sealing membrane 28.

Working fluid 38 need not flow against the first surface 42 of the sealing membrane 28 before injecting the reagent 40 since the working fluid 38 may cause the sealing membrane 28 to block the flow of the reagent 40. Applying vacuum to the reagent gap 30 may pull working fluid 38 against the first surface 42 of the sealing membrane 28. This may be prevented with a valve 68 in the inlet port 54. In one aspect, the valve 68 may be a check valve or burst valve having a cracking pressure greater than about 1 atmosphere. A burst valve may be implemented with a plug of material, such as beeswax, in the inlet port 54. In this case, the opening in the window 44 beneath the plug in the inlet port 54 may be greater than the plug to allow the plug to break free of the window 44 without impeding flow of working fluid 38. In another aspect, a burst valve may be implemented with thin impervious material that ruptures when pressure is applied to the working fluid 38. In another aspect, vacuum may be applied to the working fluid 38 while applying vacuum to the reagent gap 30 to balance out pressure forces on the working fluid 38. The working fluid 38 may be degassed before applying vacuum to prevent air bubble formation in the working fluid 38.

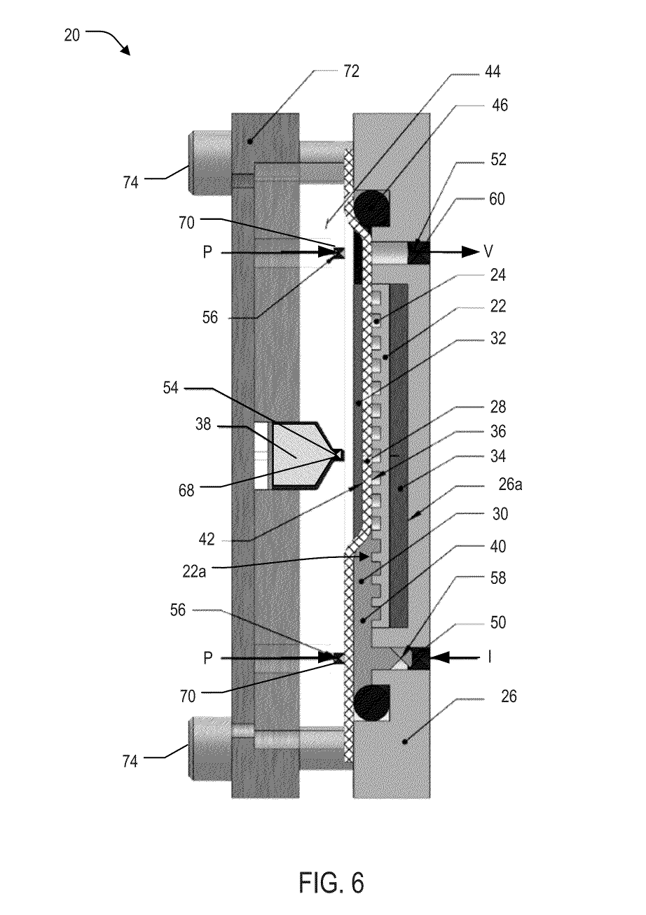

The inlet port 54 may be in the middle of the window 44 as shown in FIG. 6, or at either end of the microreactor array system 20. Further, outlet ports 56 are positioned to be generally opposing each of the inlet port 50 and outlet port 52. Further, inlet port 54 may be collocated with one of the outlet ports 56 so that there is one inlet port 54 and one outlet port 56 for the working fluid. If vacuum is applied to the reagent gap 30, prior to injecting reagent 40, then the sealing membrane 28 may be pulled down onto the upper surface 22a of the microreactor array 22. This may happen if the sealing membrane 28 is not adhered to the bottom of the window 44 or if inlet port 54 or outlet ports 56 are open to atmospheric or positive absolute pressure. If the sealing membrane 28 is pulled down, then the inertia of the sealing membrane 28 and air pressure above the sealing membrane 28 may help to fill the microreactors 24 as the advancing fill front of reagent 40 pushes the sealing membrane 28 away from the upper surface 22a of the microreactor array 22. This may impart an opposing force component towards the interior of the microreactors 24 helping to fill them completely with reagent 40. In another aspect, to prevent the sealing membrane 28 from being pulled down onto the upper surface 22a of the microreactor array 22, vacuum may be applied to inlet port 54 or outlet ports 56 in conjunction with vacuum applied to the reagent gap 30. This may balance out the pressure on the first surface 42 and the second surface 36 of the sealing membrane 28 so that the sealing membrane 28 is not pulled down onto the upper surface 22a of the microreactor array 22 prior to injecting reagent 40. In some embodiments, each of the outlet ports 56 may be provided with a suitable valve 70 to control the application of vacuum, the recovery of working fluid 38, or the like.

Outlet ports 56 may be used to flush out high viscosity working fluid 38 with another material. The temperature of the working fluid 38 or other material may be controlled, prior to injection, to control viscosity or provide thermal cycling or temperature regulation of the microreactor array 22. Lower viscosity working fluids or gasses may allow the sealing membrane 28 to be pulled up away from the upper surface 22a of the microreactor array 22. This may allow different reagents or gasses to be cycled through the reagent gap 30. This feature may be incorporated into an automated chemical processing system that cycles various reagents or gases through the microreactor array system 20. For thermal cycling, embodiments of an automated microreactor array system 20 may have built-in heaters or coolers using, for example, thermoelectric devices or channels for hot or cold liquid. In other embodiments, the entire microreactor array system 20 may be placed inside a chilling incubator or immersed in a temperature controlled liquid bath for thermal cycling or temperature regulation in general. The microreactor array system 20 may be cooled prior to injecting reagent 40 to initially slow down the chemical reactions in the microreactors 24 or to increase the viscosity of the working fluid 28. Dry gas may be pumped into the reagent gap 30 under pressure to prevent condensation if the microreactor array system 20 is cooled prior to injecting the reagent 40.

Pressurized working fluid 38 may press down on the sealing membrane 28 to seal all of the microreactors 24. To provide a good seal, the sealing pressure may be high enough so that the sealing membrane 28 conforms to the inner perimeters of the microreactors 24. Applying sealing pressure to the sealing membrane 28 with a working fluid 38 may be useful for this application. Working fluid may flow freely and conform to the shape of its container. Therefore working fluid may be used to apply uniform sealing pressure across the sealing membrane 28. By contrast, a solid material such as silicone rubber may provide uneven sealing pressure due to variations in thickness or defects in the material. Therefore, reagent may become trapped in the imperfections resulting in poor sealing and leakage in those areas. Incompressible, high viscosity working fluid 38 may be useful for displacing reagent 40 out of the way as the working fluid 38 fills the space between the window 44 and the first surface 42 of the sealing membrane 28. By comparison, gasses are compressible and have low viscosity. Accordingly, gasses may not displace reagent from the upper surface 22a of the microreactor array 22. Using a constant pressure source to inject working fluid 38 against the first surface 42 of the sealing membrane 28 may produce a seamless transition from 1) displacing reagent 40 to 2) sealing the microreactors 24. The sealing pressure source may be pneumatic, hydraulic or a mechanical spring, for example. Constant sealing pressure may be achieved via a pressure regulator, constant vapor pressure (CO₂ cartridge) or low mechanical spring rate, for example.

For some applications, chemical reactions may undergo thermal cycling. The microreactors 24 may remain sealed as the reagent 40 within the microreactors 24 expands or contracts during thermal cycling. Expansion and contraction may be exacerbated if gas is entrapped or dissolved in the reagent 40 especially if bubbles form at higher temperatures. Solubility of gas in liquid increases with pressure. Similarly boiling temperature increases with pressure. Therefore applying sealing pressure may prevent bubble formation in the microreactors 24 during thermal cycling. Degassing the reagent 40 prior to thermal cycling may also be useful. Adding substance such as glycerol or polyethylene glycol with a boiling point above about 100° C. may increase the boiling point of the reagent 40, thereby further reducing bubble formation at higher temperatures.

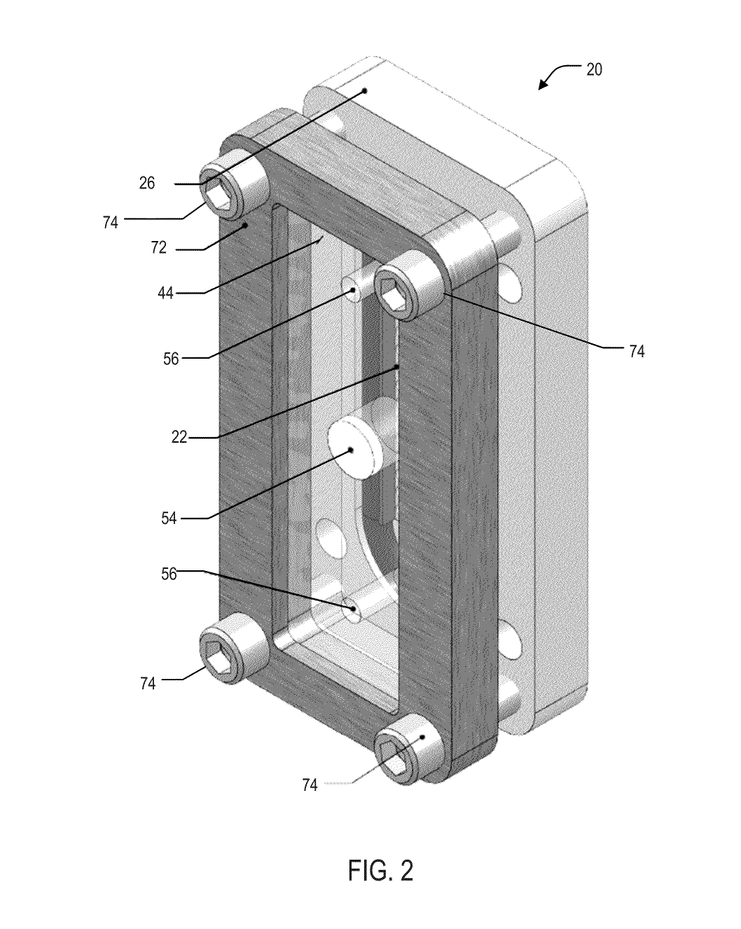

With reference to at least FIGS. 2 and 5, the microreactor array system 20 may further include a top 72 and one or more fasteners 74. In one embodiment, assembly of the microreactor array system 20 may involve providing a microreactor array 22. The microreactor array 22 may include one or more microreactors 24. Further, the microreactors may be treated with one or more reagents, functionalizing chemicals, or the like. Thereafter, the microreactor array 22 may be inserted into the cavity 26a within the base 26. Optionally, a compliant layer 34 may be positioned in the cavity 26a beneath the microreactor array 22, one or more shims 32 may be positioned adjacent the microreactor array 22, or a combination thereof. Thereafter a sealing gasket or O-ring 46 may be positioned around the microreactor array 22.

With the microreactor array 22 and sealing gasket or O-ring 46 positioned within the base 26, the sealing membrane 28 may be disposed above the upper surface 22a of the microreactor array 22. The window 44 may be positioned above sealing membrane 28 such that the sealing membrane is disposed between the window 44 and the microreactor array 22. The top 72 may be positioned over or around the window 44. With the top 72, window 44, sealing membrane 28, microreactor array 22 and base 26 positioned for assembly, one or more of the fasteners 74 may be used to couple the top 72 to the base 26, thereby providing the assembled microreactor array system 20. In one aspect, through-holes (not shown) may be positioned at intervals around a perimeter of the top 72. Similarly, the base 26 may include one or more passages or openings (not shown). When assembled, the through-holes and the openings in the base 26 may be aligned to receive at least a portion of the one or more of the fasteners 74 therein. In one example, the through-holes may be smooth cylindrical bores and the openings in the base 26 may be threaded cylindrical bores. Accordingly, a partially threaded bolt may be guided through the through-holes and then threaded or screwed into the openings in the base 26 to secure together the microreactor array system 20.

### Examples

The following Examples have been presented in order to further illustrate the invention and are not intended to limit the invention in any way.

Methods to generate nucleic acid programmable protein arrays (NAPPA) using embodiments of a microreactor array system according to the present disclosure are provided. Methods to screen for antibodies in patient serum samples and analyze the results are also provided.

For the fabrication of microreactor arrays, a photolithography mask for a microreactor array was designed using computer aided design (CAD) software (AutoCad, Autodesk, San Rafael Calif.). A 150 mm diameter circular outline was drawn. Seven microscope slide format slides 25.4 mm×76.2 mm were spaced apart within the circular outline. 50 μm wide cut-lines ‘streets’ were added between slides for dicing. For each slide, about 14,000 130 μm diameter circles were drawn for the microreactors in a hexagonal closest packing array pattern with 375 μm center-to-center spacing leaving at least 1.33 mm empty areas, without features, at the top and bottom of each slide. These areas were used for autofocus by the microarray scanner. Numbers were placed along the sides and bottom of each slide to identify rows and columns respectively. A logo was included along the bottom of each slide to orient it during the various processing steps of printing, assaying and scanning. 200 μm diameter circles were added outside of the slides for depth measurement during the etching process.

To fabricate photolithography masks for the microreactor array, a photolithography mask drawing was provided to a mask manufacturer (JD Photo-Tools, Oldham UK). ‘7″×7″ chrome glass’, ‘super-high resolution’ (128K dpi), ‘darkfield’ and ‘design viewed from glass side’ were specified. Clear areas of the mask (no chrome) correspond to the etched areas of the silicon wafer in subsequent steps.

To pattern silicon wafers, 6″ (150 mm) silicon wafers (University Wafer, Boston Mass.) with standard thickness of 675 μm+/−25 μm were obtained. One side of each wafer was coated with 300 nm LPCVD low stress nitride. Nitride was spin coated with 1 μm AZ 3312 (AZ Electronic Materials Inc., Branchburg N.J.) positive photoresist. Samples were then baked at 100° C. for two minutes. Photo resist was exposed with the photolithography mask on mask aligner (OAI, San Jose Calif.), developed in AZ300 MIF developer (AZ Electronic Materials Inc., Branchburg N.J.) for 45 seconds, and hard-baked at 100° C. for 2 minutes. Nitride film was selectively etched with reactive ion etch (RIE) and photoresist was removed with acetone.

To etch microreactors, HNA etchant mixture of 49% hydrofluoric acid (HF), 70% nitric acid (HNO3) and (>98%) glacial acetic acid (CH3COOH) in the ratio of 2.75:1.75:1 was prepared. Wafers were isotropically etched for about 30 minutes to 70 μm depth and 270 μm diameter. Etching silicon with HNA is exothermic so the wafers were agitated to maintain uniform temperature.

To grow an oxide layer, the surface of the wafer was cleaned in piranha mixture (1:1 mixture (sulfuric acid (H₂SO₄)):(hydrogen peroxide (H₂O₂))) for 15 minutes. Surface was cleaned in buffered oxide etch of hydrofluoric acid (HF) and ammonium fluoride (NH₄F) (1:6 mixture of HF:NH₄F) for 10 seconds. 95 nm thin film silicon dioxide (SiO₂) layer was grown at 1,000° C. for about 3 hours in an oxygen furnace (Tystar 4600, Torrance Calif.).

Wafers were diced along cut-lines (Advotech Company, Inc., Tempe Ariz.) into 7 individual microreactor array slides. Saw blade kerf was 50 μm so actual slide dimensions were 25.35 mm×76.15 mm.

To functionalize surfaces, residual organic materials were cleaned from the microreactor array slides in piranha (1:1 mixture of H₂SO₄:H₂O₂) for 30 minutes. After rinsing with DI water and drying with compressed air, slides were immersed in a 2% solution of (3-Aminopropyl) triethoxysilane (APTES) in acetone for 30 minutes. Slides were thoroughly rinsed in acetone and DI water, and dried with compressed air.

To print DNA into microreactors, first plasmid DNA was obtained for a set of genes from the DNASU Plasmid Repository (Center for Personal Diagnostics, Biodesign Institute, Arizona State University, Tempe Ariz.). Clones were sequence verified and inserted in the E. coli open-dual recombination cloning vector open reading frames (ORFs) with the natural stop codon absent and a GST-tag appended to C-terminus.

To purify plasmid DNA, E. coli colonies of clone vectors were cultured and harvested. Plasmid DNA was purified from the harvested cultures with a 96 microreactor miniprep (Whatman Filter Plates, Sigma-Aldrich, St. Louis Mo.). Miniprep DNA was transferred to a 384-well microplate. Miniprep plasmid DNA was normalized to 100 ng/μl (Nanodrop 8000, Thermo Scientific, Wilmington Del.) and stored at 4° C. until printing.

To prepare a 1×NAPPA printing-mixture, components were thawed on ice and mixed together in the following order and proportions: 93% nuclease-free DEPC-treated water (Ambion, Life Technologies, Grand Island N.Y.), 0.6% BSA (Sigma-Aldrich), 1% anti-GST antibody (GE Healthcare), 5% BS3 cross-linker (Thermo Scientific, Pierce). The printing mixture was aged at 4° C. for one day to allow partial cross-linking of BS3, and then aliquoted into a 384-well microplate for printing. The printing mixture was stored on ice at all times throughout the process.

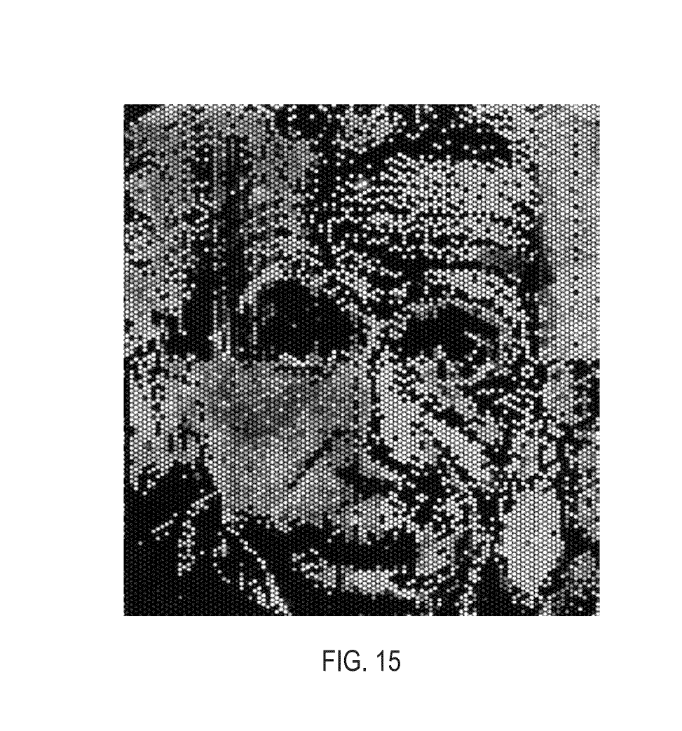

To prepare the bitmap pattern for FIG. 15, a digital RGB image was resized to 108×108 pixels using function imresize( ) (Matlab, MathWorks, Natick Mass.). The Matlab function imrgb2ind( ) was used to reduce the image to an indexed image with 5 uniformly spaced grayscale intensities, gray(5). The indexed image was then split into two halves to fit on a 1″×3″ microscope slide format and converted to a bitmap file using Matlab function imind2rgb( )

The APTES functionalized microreactor array slides were aligned on the deck of a non-contact piezoelectric dispensing microarrayer (PiXY, Engineering Arts, Tempe Ariz.) to prepare them for printing. To print microreactor arrays, piezoelectric dispensers (Engineering Arts) were used on the microarrayer. Dispensers were primed with DI water. The 384-well microplate with printing-mixture was placed on the deck of the microarrayer and 2 μl printing-mixture was aspirated into each dispenser using on-head aspiration syringes. Twelve 0.1 nl drops of printing-mixture were dispensed in short bursts at 12,500 drops-per-second into microreactors using the predefined microarray pattern defined above. Non-contact piezoelectric on-the-fly dispensing at uniform print-head speed of 175 mm/sec was used. The piezoelectric tips were cleaned by flushing DI water through the dispenser while the dispenser tip was submerged in flowing DI water. Within 10 minutes, the same process was repeated with the 384-well microplate of plasmid DNA. Three drops of plasmid DNA was printed in the same spots as the printing-mixture using the same aspirating and dispensing parameters (12,500 drops-per-second) as for the printing-mixture.

To print the microarray for the image shown in FIG. 15, DNA and printing mixture were combined and printed onto a microreactor array using a PiXY microarrayer capable of printing colored bitmaps.

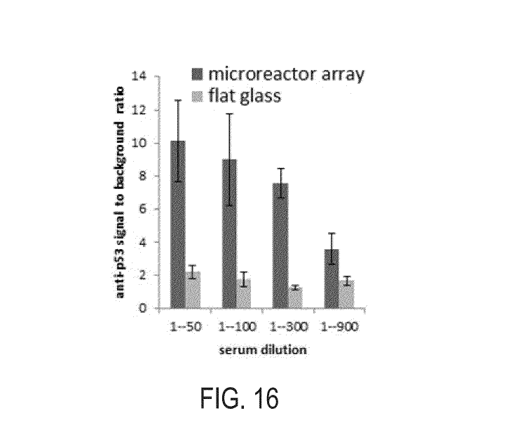

To print flat glass slides for p53 serum screening comparison in FIG. 16, DNA and printing mixture were combined and printed on APTES coated glass microscope slides (Fischer Scientific, Waltham Mass., Cat. No. 12-544-1) using Genetix QArray2 pin-spotter at 2,500 spot density per microscope slide. Printed plasmid DNA microarrays were stored at room temperature in a sealed container with desiccant.

For protein expression, microreactor array slides were first blocked. This step washed away unbound printed molecules from the slides' surfaces. A centrifuge (Beckman Coulter model Allegra X-15R, Indianapolis Ind.) was programmed for 3750 RPM, maximum acceleration and maximum deceleration. Slides were submerged in a tray containing 8 ml of Superblock Blocking Buffer in TBS (Thermo Scientific product #37535B, Rockford Ill.). Samples were accelerated up to the maximum speed and decelerated right away. Slides were rinsed in DI water and dried with compressed air.

The same procedure as outlined above for microreactor array slides was used to block flat glass slides for FIG. 16. The centrifuge was not required. Slides were blocked for one hour on a rocking table (VWR model 200, Radnor Pa.).

In vitro coupled transcription and translation (IVTT) reagent was prepared from the “1-step human in vitro protein expression kit” (Thermo Scientific, Rockford, Ill.). The four components of the kit and were thawed and stored on ice. Components were mixed in the following order and proportions: 34% HeLa lysate, 10% accessory proteins, 22% reaction mix and 34% nuclease-free water, using 60% HeLa lysate compared to the normal recipe. Mixture was stored on ice. Mixture was degassed in a vacuum desiccator until bubbles were gone (approximately 5 minutes).

IVTT reagent was applied to microreactor array slides by first inserting a microreactor array slide into a microreactor array system according to the present disclosure. An O-ring was positioned around the microarray slide, and a sealing membrane was placed on top of the O-ring and slide. A window was placed on top of the sealing membrane and the microreactor array system was clamped together using fasteners. Vacuum was applied to the reagent outlet port and 300 μl IVTT reagent was injected into the reagent inlet/outlet port. Pressurized working fluid was injected into the inlet port in the window to displace the reagent and seal the microreactors.

To apply IVTT reagent to flat glass slides for FIG. 16, a gasket (Hybriwell Sealing System item 440904, Grace Biolabs, Bend Oreg.) was attached to the slide. Using a manual pipette, 150 μl IVTT reagent was injected into the hole at one end of the gasket.

Flat glass slides and gasket were incubated at 30° C. for 1.5 hours and then 15° C. for 0.5 hours (EchoTherm chilling incubator, Torrey Pines Scientific, Carlsbad Calif.). The assembled microreactor array system was incubated at 30° C. for 2 hours and then 15° C. for 1 hour. Incubation times were longer than standard NAPPA on flat glass slides to accommodate longer heating and cooling times due to the higher thermal mass of the microreactor array system.

To detect fluorescent signals, proteins were first fluorescently labeled. To preserve molecular functionality, slides were not allowed to dry out between processing steps. A 5% milk-PBST (0.2% Tween) blocking buffer was prepared by combining 500 ml 1× phosphate buffered saline (1×PBS) with 25 grams 100% instant nonfat powdered dry milk and 1 ml Tween 20 detergent and mixed with magnetic stirring bar for 10 minutes. The resulting solution was stored at 4° C. A small tray was filled with blocking buffer. The microreactor array system was disassembled and the slide was removed and submerged into blocking buffer. Blocking was performed for one hour at room temperature on a rocking table (VWR model 200, Radnor Pa.) with blocking buffer replaced three times. A primary label was prepared by mixing 10 μl mouse anti-GST monoclonal antibody (Cell Signaling Technologies, Danvers Mass.) in 3 ml blocking buffer. Slides were incubated in primary label overnight at 4° C. on a rocking table then rinsed with blocking buffer 3 times for 10 minutes each, replacing blocking buffer each time. Secondary label was prepared by mixing 6 μl Alexa Fluor 647 nm goat anti-mouse IgG antibody (Life Technologies, Grand Island N.Y.) in 3 ml blocking buffer. Slides were incubated in secondary label for 1 hour at room temperature on a rocking table in the dark to prevent photo bleaching of the fluorescent dye. Rinsing buffer was prepared by mixing 500 ml 1×PBS and 1 ml Tween. Slides were rinsed in rinsing buffer three times for 1 minute each time, replacing the rinsing buffer each time. Slides were rapidly rinsed in DI water 6 times replacing water each time and then dried with compressed air and stored in the dark, at room temperature with a desiccator.

To probe serum samples for FIG. 16, following protein expression, flat glass slides were blocked in 5% milk-PBST (0.2% Tween) on a rocking table at room temperature for 1 hour. Slides were incubated with diluted serum sample in proplate 4-well tray set (Grace Bio-Labs, OR) at 4° C. overnight. Slides were then washed in 5% milk-PBST (0.2% Tween) three times for 5 min each time. Protein display was detected with Alexa Fluor 647 labeled goat anti-human IgG secondary antibody (Jackson ImmunoResearch, West Grove, Pa.). Finally, slides were washed in DI water and dried with compressed air.

Microreactor array slides were imaged by first programming a fluorescent microarray scanner (PowerScanner, Tecan, Mannedorf Switzerland) for microreactor array slides. Coordinates of the empty areas at the top and bottom of slides were defined for autofocus along with the reflectivity of those areas. A 70 μm offset was defined for microreactor depth after autofocus. Self-adhesive 325 μm thick backing was applied to slides to accommodate the 1 mm slide thickness requirement of the scanner. Slides were scanned at 10 μm resolution, 25% laser power (out of 30 mW) and 25% (out of 800% maximum) photo multiplier tube (PMT) detector gain. Data was extracted (Array-Pro, Media Cybernetics, Rockville Md.) and analyzed (Excel, Microsoft, Redmond Wash.). With reference to FIG. 16, signal to background ratio of anti-P53 response was plotted. Signal for each dilution point was the average signal of six spots on the array, and signal of each spot was the median pixel value of that spot. Background was the median signal of all of the spots on the array.

2. Microreactor Array System with Automation and Detection

In one aspect, the present disclosure combines microplate and microarray technology. For example, microarray-scale well plates (i.e., microreactor arrays) may be used to carry out tens of thousands of independent reactions simultaneously. By comparison, the present disclosure may differ from conventional ‘lab-on-a-chip’ devices (i.e., miniaturized, serial, two-dimensional networks of micro-channels emulating electrical circuits or conventional chemical processing plants). Instead, a microreactor array system may have two independent, yet complimentary, fluid paths separated by a flexible sealing membrane. The fluid paths may be paper thin (e.g., about 100 microns thick). A first pathway may access an array of thousands of microreactors to fill them with reagent in parallel for high density chemical processing. A working fluid on the opposing side of the sealing membrane may push against the reagent through the sealing membrane.

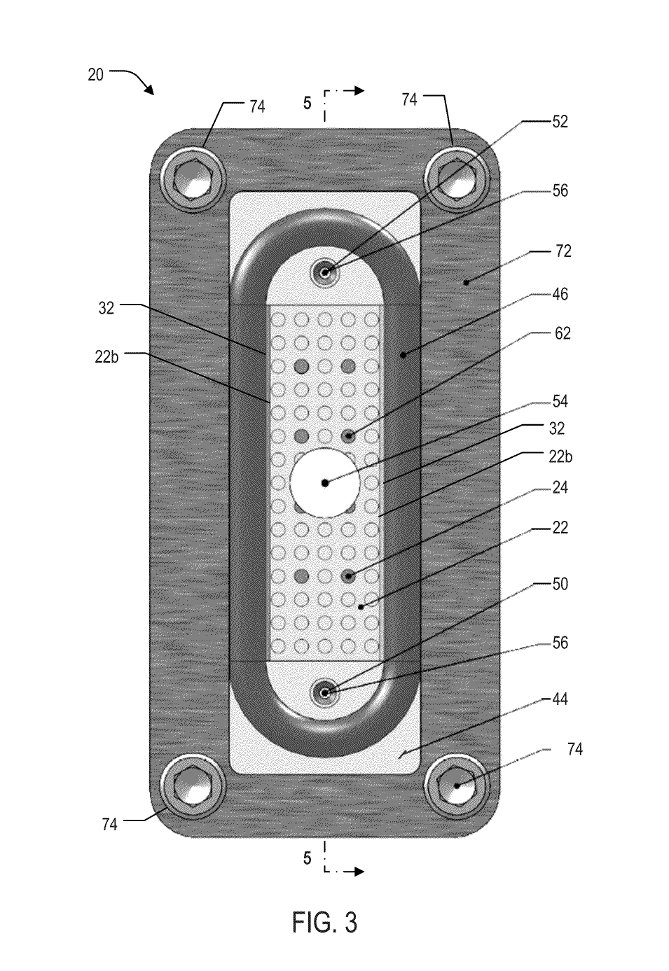

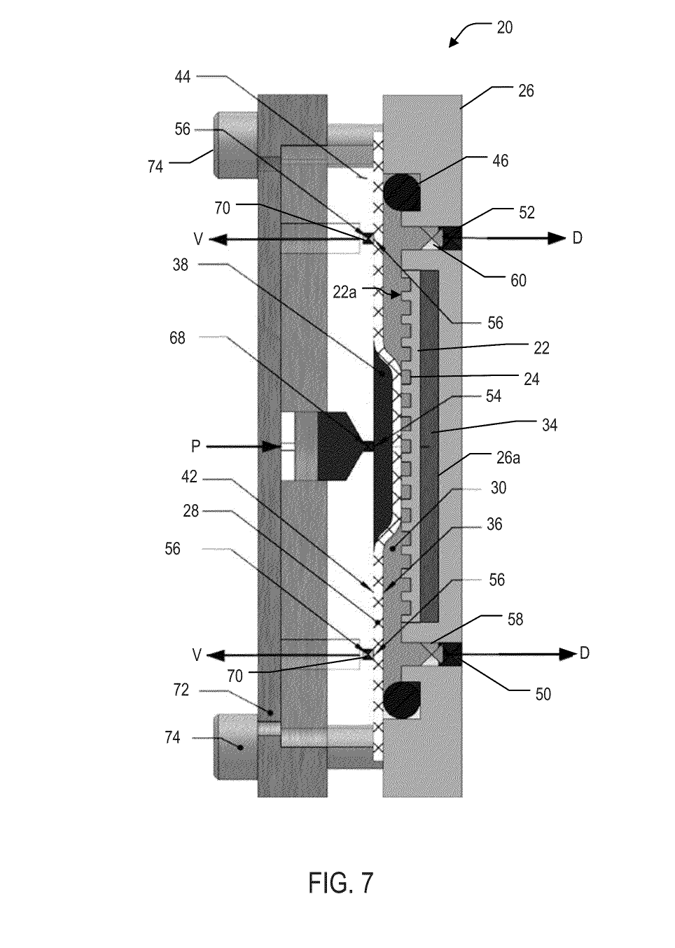

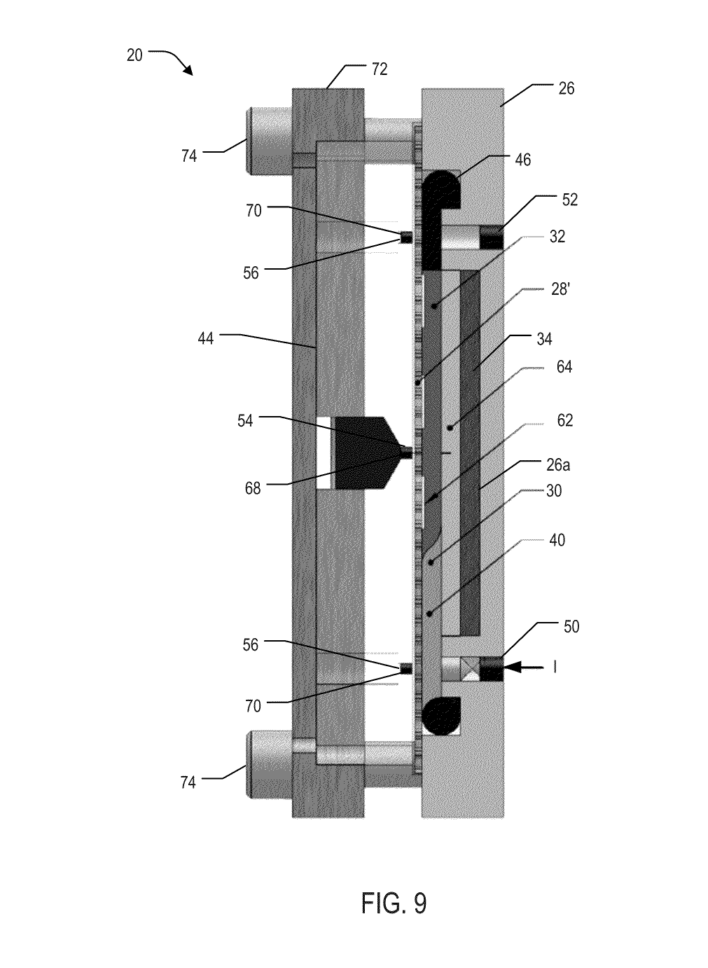

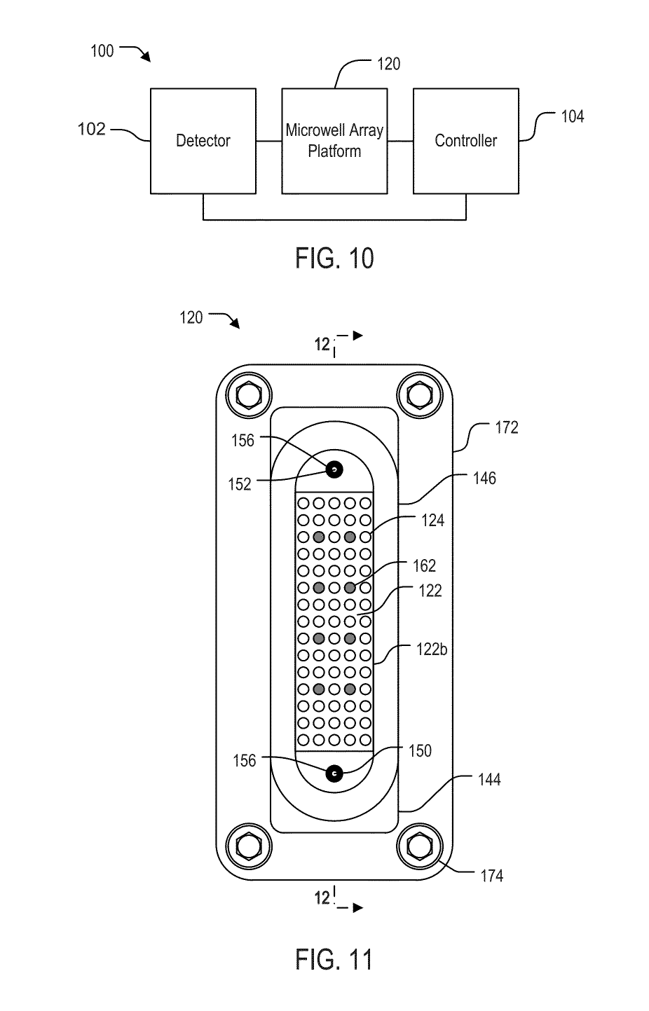

Turning now to FIG. 10, an embodiment of a system 100 according to the present disclosure includes a detection subsystem or detector 102, an automation subsystem or controller 104, and a microarray system 120. In one aspect, the microarray system 120 may be similar to the microarray system 20 illustrated in FIGS. 1-9. Each of the components of the system 100 may be in communication with each of the other components. For example, detector 120 may be in communication with both the controller 104 and the microreactor array system 120. Accordingly, the controller 104 may be configured to automatically control the detector 120 to detect a characteristic of the microreactor array system 120.

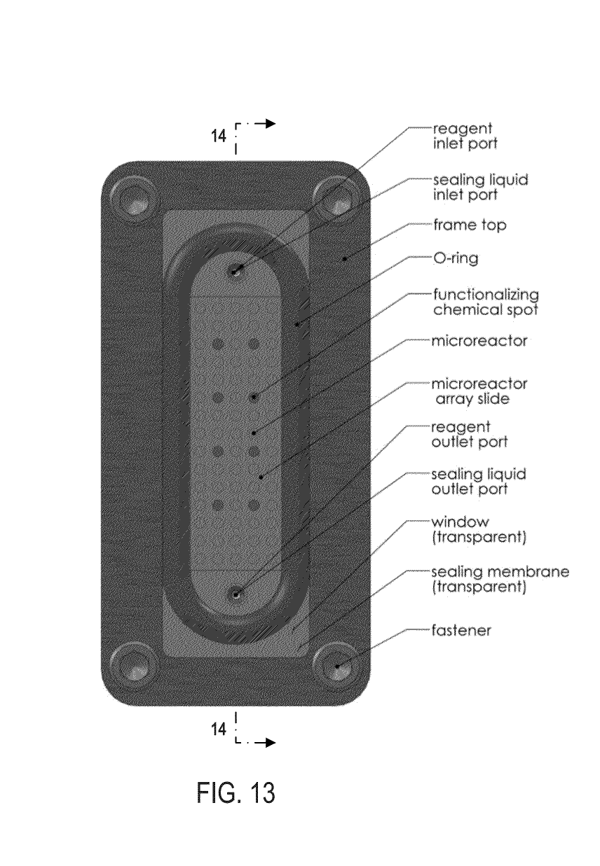

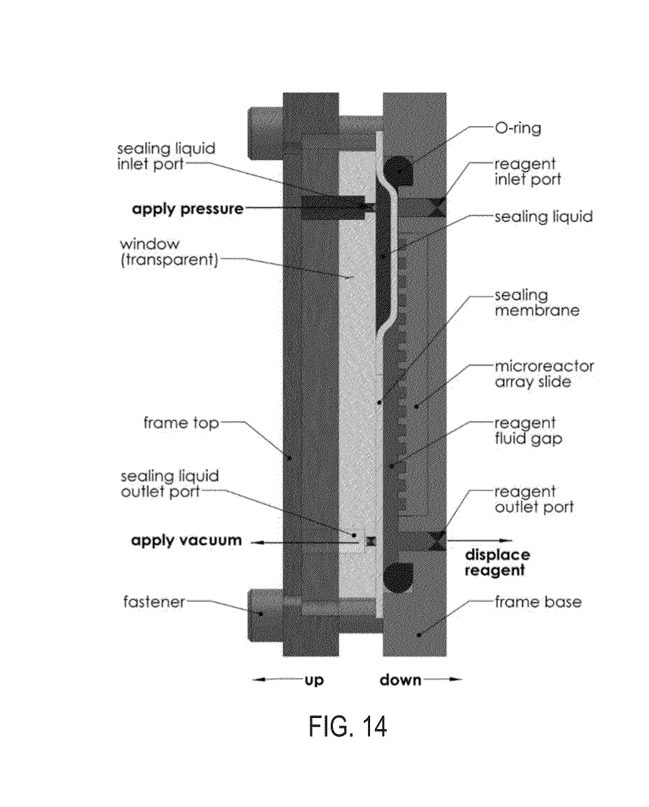

With reference to FIGS. 11-14, the microreactor array system 120 may include microreactor array 122 having one or more microreactors 124 formed in a surface 122a of the microreactor array 122. Additionally (or alternatively), the microreactor array 122 may be a smooth surface unto which materials such as nucleic acids, enzymes, or other chemical reagents may be spotted, printed, or otherwise deposited. The microreactor array system 120 may further include a base 126 having a cavity 126a for housing the microreactor array 122. When positioned within the cavity 126a, a sealing gasket or O-ring 146 may be positioned about a perimeter of the microreactor array 122. Thereafter, a sealing membrane 128 may be positioned over the surface 122a of the microreactor array 122 followed by a window 144 and a top 172. The sealing membrane may have an array of flexible microreactors formed on its second surface in contact with the surface 122a of the microreactor array 122. The assembled components of the microreactor array system 120 may be fastened, clamped, or otherwise held together with one or more fasteners 174.

As in the case of microreactor array system 20, the microreactor array system 120 may include various inlets, outlets, valves, ports, or other like features for delivering various fluids, chemicals, reagents, gasses, or other like materials to either a reagent gap 130 disposed between the surface 122a and a second surface 136 of the sealing membrane 128, a first surface 142 of the sealing membrane 128, or a combination thereof. For example, the microreactor array system 120 includes an inlet port 150 and an outlet port 152 being associated with a valve 158 and a valve 160, respectively. The inlet port 150 and the outlet port 152 may be in fluid communication with the reagent gap 130. For example, a reagent 140 may be injected through the inlet port 150 (or the outlet port 152) and into the reagent gap 130 in order to fill the microreactors 124 with reagent 140. In another aspect, a set of ports 156 and valves 170 may be in fluid communication with the first surface 142 of the sealing membrane 128. In one example, a working fluid 138 may be injected through at least one of the ports 156 in order to displace the sealing membrane 128 towards the upper surface 122a of the microreactor array 122 in order to seal off the individual microreactors 124 in a manner similar to that described for microreactor array system 20.

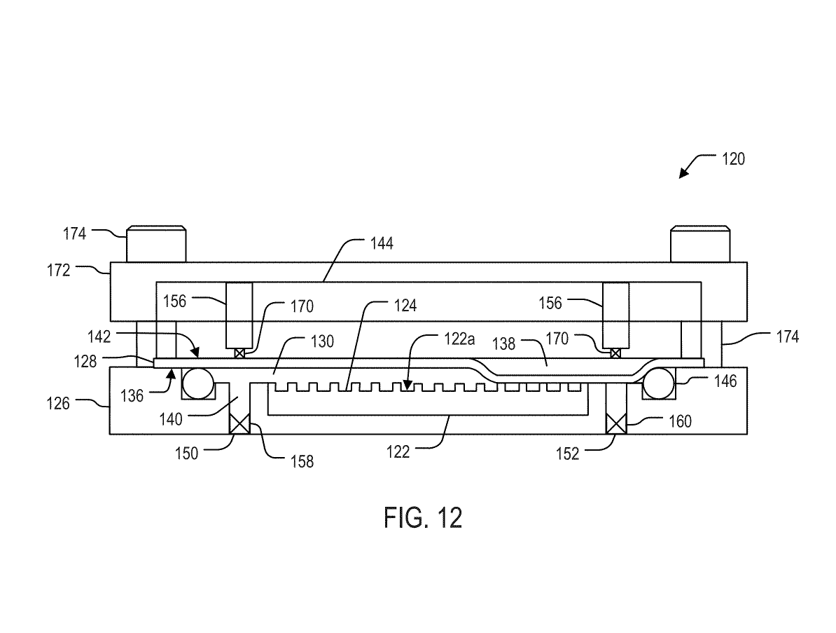

Notably, the microreactor array system 120 may be generally free of ports, valves, or other like features positioned above the surface 122a of the microreactor array 122 (see, e.g., inlet port 54 in microreactor array system 20). In one aspect, it may be useful to have an unobstructed path in order to use various analytical equipment with the microreactor array system 120. For example, a detector 102 may include a fluorescence microscope or camera system for illuminating the microreactor array 122 and detecting a corresponding output. If the microreactor array system includes valves, ports, or other like features, these features may interfere with or otherwise obstruct the aforementioned illumination or detection procedures. Accordingly, working fluid 138 may be provided to the first surface 142 of the sealing membrane 128 through at least one of the ports 156 as illustrated in FIG. 12.

While one possible use for the working fluid 138 is to displace reagent and seal the microreactors 124 into isolated chemical reaction chambers, this can also be reversed. For example, reagent 140 or another reagent may be used to press against the second surface 136 of the sealing membrane 128 and displace the working fluid 138. Accordingly, embodiments of the system 100 may provide for multi-step chemical processing involving repeated cycles of washing, blocking, addition of reagent, and sealing or resealing of the microreactors 124. In another aspect, working fluid 138 may be actuated or pulsed repeatedly during chemical processing to aid in flushing out reagent 140, providing mixing or agitation, or a combination thereof. For example, it may be useful to provide agitation within the microreactors 124 during primary and secondary antibody labeling for immunoassays. In another aspect, this can be reversed so that reagent 140 may be actuated or pulsed repeatedly to aid in flushing out working fluid 138, providing mixing or agitation, or a combination thereof. Therefore, the flexible sealing membrane 128, working fluid 138 and reagent 140 may provide expanded fluidic and chemical processing capabilities.

In one embodiment, the microreactor array system 120 may be operated manually or automatically. In the case of manual operation, the microreactor array system 120 may process the first step of a nucleic acid programmable protein array (NAPPA) assay (i.e., protein expression). Subsequent steps of washing, blocking, screening and antibody labeling may be done by hand using standard wet lab bench immunoassay protocols. In another embodiment, the present disclosure provides a fully automated instrument for multi-step assay processing of microreactor arrays. The controller 104 may provide repeatable processing parameters of volume, flow rate, temperature, reagent concentrations, incubation times, washing and blocking protocols, and drying protocols. Consistent processing conditions may produce more consistent assay data compared to manual processing. The controller 104 may also reduce the occurrence of contamination or equipment failure due to manual handling. As multi-step assays are common in the life sciences, the disclosed system and method may facilitate high-throughput, automated, multi-step assays in sealed microreactors containing untethered molecules. The controller 104 may further enable applications such as automated, high-throughput post translational modification (PTM) of proteins.

In one aspect, integrating the detector 102 into the system 100 including the automated microreactor array system 120 may provide a standalone, high-throughput, multiplexed, end-to-end, processing system. For example, the detector 102 may include a fluorimeter, colorimeter, or luminometer coupled to (or otherwise in communication with) the microreactor array system 120. The combination of a detector 102 and the microreactor array system 120 may allow for real-time monitoring of chemical reactions in sealed microreactors.

In some embodiments, the detector 102 may include an integrated processing and detection instrument. In one example, an integrated processing and detection instrument may enable coupled NAPPA-ELISA assays. This may combine advantage of enzyme-linked signal amplification of ELISA together with the convenience and utility of NAPPA to express thousands of fresh functional protein antigens from printed DNA microarrays. Compared to printing proteins directly, DNA is generally easier to print using piezoelectric dispensing techniques. In one aspect, DNA is typically stable and in some cases, may be stored for months at room temperature prior to assay with negligible degradation. By comparison, conventional ELISA kits may include relatively unstable pre-coated protein antigens that are dried out prior to assay, possibly resulting in conformational and functional degradation. Further, the disclosed NAPPA-ELISA method may be used to assay thousands of analytes simultaneously as compared to typical ELISA kits that may have less that about a dozen analytes per 96-well plate. The coupled NAPPA-ELISA assay may further provide improved detection limits for target analytes such as cancer autoantibody biomarkers in blood serum. For example, decreasing reaction volumes may improve sensitivity and reaction time compared to standard ELISA in microplates 25. Monitoring fluorogenic substrate signal amplification in real time may improve quantitation as in qPCR vs. PCR. Furthermore the fluorescent signal from liquid in microreactors may be brighter and more uniform than dry microreactors providing higher sensitivity and better signal quantitation. Integrated detection may further provide end-to-end hands-free automation from initially setting up the instrument with reagents and samples to gathering data.

With reference to FIG. 15, an example of the use of a microreactor array system 120 and detector 102 for protein expression and detection is shown. In one aspect, the expression of fluorescently labeled protein in microreactors illustrates that at least about 10,000 protein spots may be expressed on a single microreactor array with little or no diffusion or evaporation during a three hour period of incubation.

In general, a system according to the present disclosure may provide end-to-end processing of microreactor arrays together with integrated optical detection. The combined microreactor array system with integrated detection may be used to carry out NAPPA-ELISA. In one example, NAPPA-ELISA may be used to detect anti-P53 autoantibody biomarker in the serum of a patient that has tested positive for cancer (see FIG. 16). The assay may have better sensitivity and selectivity than a system lacking integrated detection.

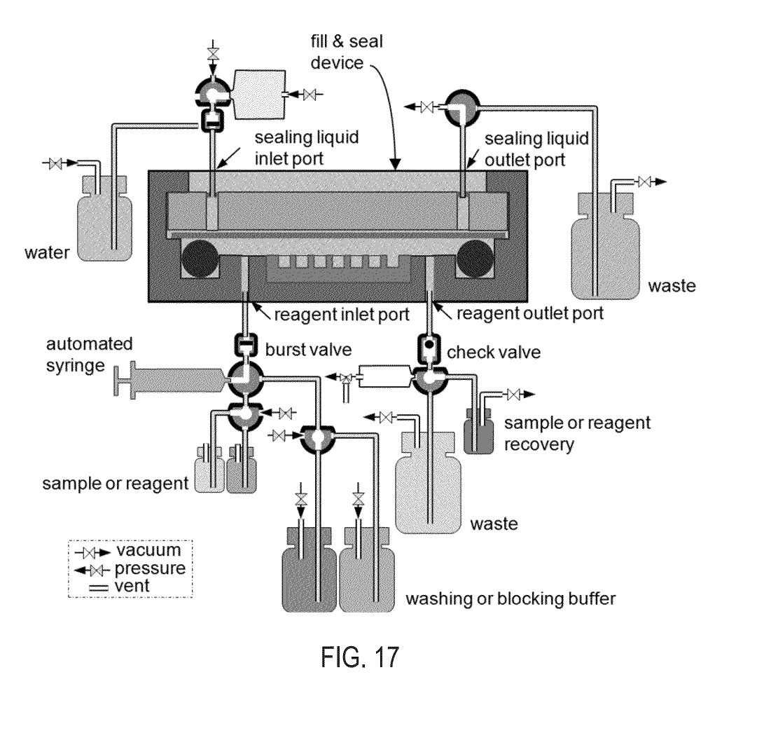

FIG. 17 is a schematic of a chemical processing system to sequentially refill an array of microreactors with various fluids and reseal them into isolated microreaction chambers. Sample fluids, reagents, washing buffers, blocking buffers, vacuum or air may be injected into the microreactors through a regent inlet port and removed through a reagent outlet port underneath the microreactor array system. Working fluid, water, vacuum or air may be injected onto a sealing membrane through a working fluid inlet port and removed through a working fluid outlet port on top of the microreactor array system. An automated syringe may inject sample or reagent into microreactors. The other fluids may be transported by pressure or vacuum actuated by solenoid valves. Selector valves at each of the ports may switch between different fluids. Valves may be actuated electronically under computer software control.

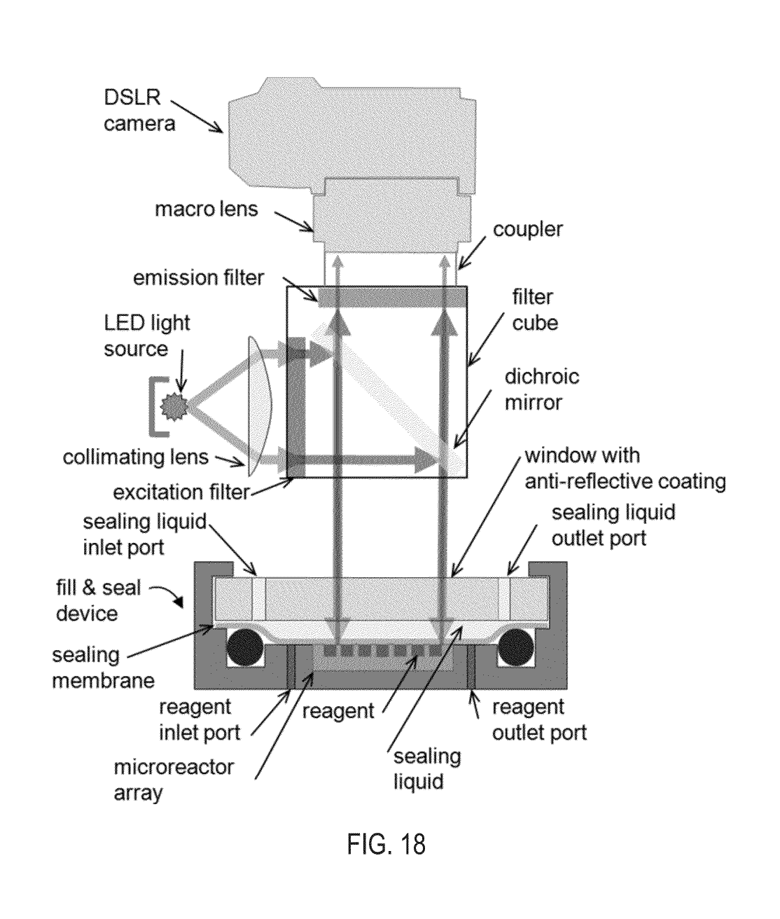

FIG. 18 is a schematic of an epifluorescent detection system for an array of microreactors within a microreactor array system. A transparent window with anti-reflective coating may transmit light to and from the microreactor array. The microreactor array may be illuminated by collimated light from a light emitting diode (LED) light source through a filter cube. An excitation filter may transmit light of a specific wavelength. A dichroic mirror may reflect shorter wavelength light onto the microreactor array causing fluorescent chemicals in the microreactors to emit longer wavelength light that is transmitted through the dichroic mirror and emission filter and detected by a digital single lens reflex (DSLR) camera.

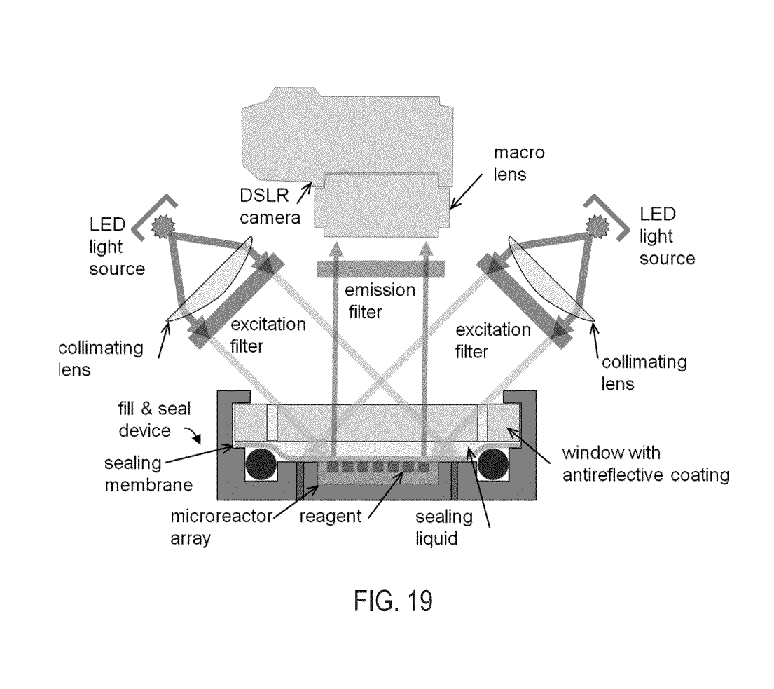

FIG. 19 is a schematic of an alternate illumination scheme for fluorescent signal detection of an array of microreactors within a microreactor array system. Two LED light sources may illuminate the microreactor array at oblique angles. Light from each LED may pass through a collimating lens, excitation filter and window to illuminate the microreactor array. Fluorescent chemicals in the microreactors may emit longer wavelength light that passes through an emission filter and is detected by a DSLR camera.

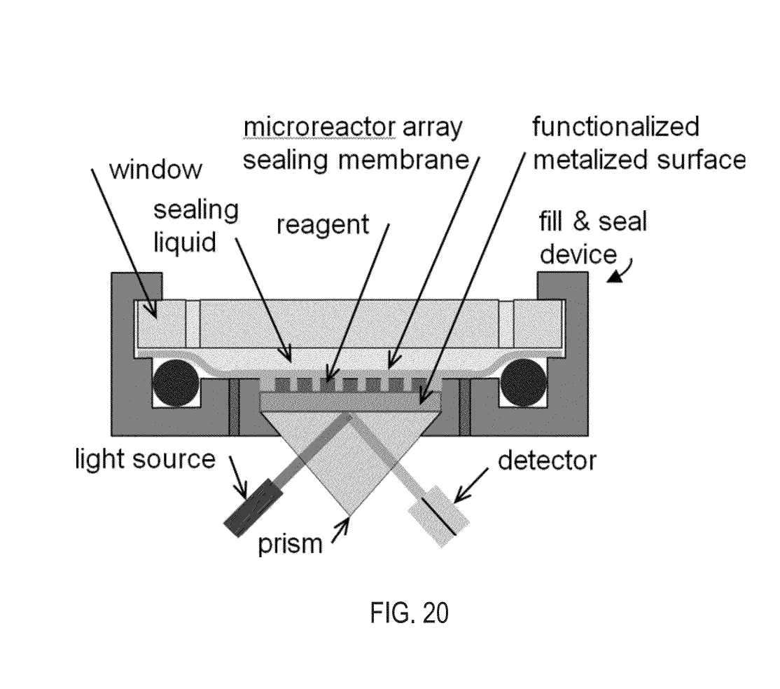

FIG. 20 is a schematic of a system to detect molecular binding to a functionalized metal surface using surface plasmon resonance (SPR) optical detection integrated into a microreactor array system. Isolated chemical reactions may take place in an array of microreactors on a flexible sealing membrane that comes in contact with a functionalized metalized surface. The bottom of the metalized surface may be illuminated by light passing through a prism. The light excites surface plasmon resonance on the metalized surface which absorbs the reflected light at a specific angle observed by the detector. Molecules bound to the metalized surface may change the surface plasmon resonance frequency which changes the absorbance angle. The system may monitor real time binding of molecular products in isolated chemical microreactions. Alternately, it may monitor kinetic binding of other molecules to the molecular products by retracting the sealing membrane and flowing other chemicals over the metalized surface.

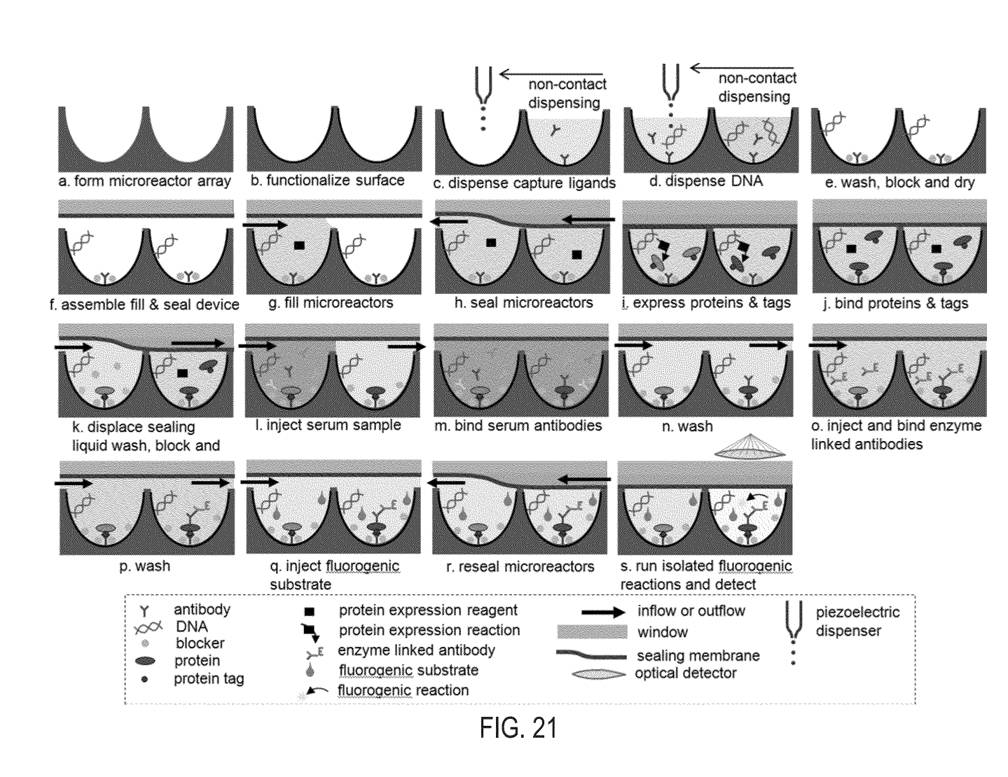

FIG. 21 is a schematic of an example biochemical immunoassay implemented within an integrated microreactor array processing system. FIG. 21 depicts NAPPA-ELISA that combines in situ protein expression of the nucleic acid programmable protein array (NAPPA) with the fluorogenic signal amplification of enzyme-linked immunosorbent assay (ELISA). The individual panels in FIG. 21 show the step-by-step protocol of the assay in terms of refilling and resealing microreactors. Molecular interactions in the microreactors are depicted symbolically. Moving sequentially through the panels in FIG. 21: a. form an array of microreactors; b. functionalize the microreactors' surfaces; c. dispense capture ligand functionalizing chemicals into microreactors; d. dispense DNA functionalizing chemicals into microreactors; e. wash away unbound functionalizing chemicals and dry the microreactor array; f. assemble the microreactor array system; g. inject reagent to fill microreactors; h. inject working fluid to seal microreactors; i. express proteins together with their tags; j. bind protein tags to their capture ligands; k. displace working fluid, wash away unbound molecules and block microreactors' surfaces; l. inject serum sample; m. bind serum antibodies; n. wash away unbound antibodies; o. inject and bind enzyme linked antibodies; p. wash away unbound antibodies; q. inject fluorogenic substrate; r. inject working fluid and reseal microreactors; s. run isolated enzymatic reactions and detect fluorescent signals.

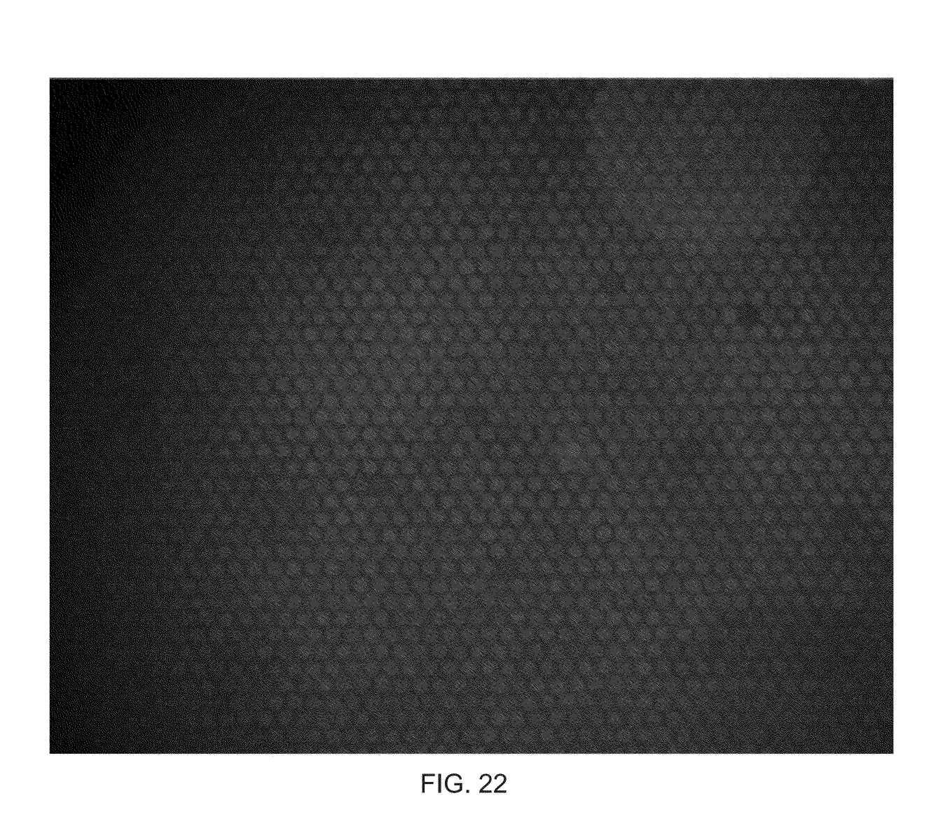

To confirm feasibility of the invention, a fluorescent image of microreactors inside of an integrated microreactor array system was acquired. See FIG. 22.

With respect to multi-step chemical processing, it may be useful to provide effective displacement of one fluid with another in the microreactor array format as opposed to a flat surface. For example, during protein expression for NAPPA, IVTT reagent may be injected in a reagent gap 130 and then high viscosity pressurized working fluid may be used to seal the microreactors through a flexible sealing membrane. After expression, IVTT reagent may be flushed out of the microreactors with a blocking solution. This entails first releasing the sealing membrane from the surface of the microreactor array surface. The flexible sealing membrane may operate within its elastic limit so that there is no permanent deformation of the sealing membrane as it traverses back and forth across the reagent gap. However, it may be useful to first replace the high viscosity working fluid with unpressurized lower viscosity working fluid like water and or dry gas to release the sealing membrane from the microreactor surface. Generally, lower viscosity liquid may not displace higher viscosity liquid as a single continuous sheet. Instead the lower viscosity liquid may flow in channels through the higher viscosity fluid. Therefore, relatively large volumes of lower viscosity working fluid may be required to displace all of the viscous fluid by convection (i.e. advection combined with diffusion). Flushing out high viscosity working fluid may be assisted by periodically pulsing the reagent on the opposite side of the flexible sealing membrane. This way the lower viscosity working fluid and reagent may help flush each other out across the flexible sealing membrane.

In another aspect, a microreactor array system may facilitate working with small volumes of patient sample or reagents. Following a blocking step in a typical ELISA procedure, the blocking solution may be replaced with less than about 500 microliters of patient sample or label antibody reagent, depending on the assay. However for displacement of blocking solution from microreactors, flow rates, reagent gap thickness and viscosity may be carefully monitored with respect to dimensionless Péclet, Reynolds and Schmidt numbers to characterize laminar vs. turbulent flow and advection vs. diffusion to achieve effective displacement. Effective small volume fluid displacement may be validated for microreactor arrays by demonstrating uniform detection signal from one end of the microreactor array to the other. Agitating reagent back and forth while pulsing the complimentary working fluid may help to evenly distribute patient sample or reagent across the microreactor array surface. Small dead-volume valves may be used at the reagent inlet and outlet ports to minimize volume required for assay. A separate reagent inlet port for small volumes may help reduce dead volume.

In some embodiments, protocols may include thermal cycling at various temperatures. One example protocol for NAPPA includes cycling between about 30° C. for protein expression, about 15° C. for protein capture, about 4° C. for primary labeling, and about 21° C. for secondary labeling. Assays may be thermally controlled with a temperature regulated, circulating water bath to provide rapid heating and cooling and uniform temperature across the microreactor array. Valves may switch water from different baths at different temperatures to rapidly change the temperature of the assay.

In some embodiments, the present system integrates real time optical detection. In one example, a microreactor array system may include a high quality glass window (e.g., Schott Borofloat 33). The window may have two machined holes with plastic inserts for inlet and outlet ports for the working fluid. Window thickness may be selected to maintain structural integrity, optical clarity and allow for higher numerical aperture optics by reducing working distance through smaller window thickness.

Furthermore, the system may be fitted with a transparent sealing membrane. A sealing membrane made from polyester (Mylar) may have a relatively high autofluorescence. Accordingly, embodiments of the system may include sealing membranes with lower autofluorescence selected from PMMA (acrylic), polycarbonate, polystyrene or polyolefin, or combinations thereof or other materials.

In addition to having greater optical clarity than materials such as polycarbonate, it may be useful to provide a glass window to satisfy fluidic in addition to optical requirements. In one aspect, glass may be 20 times stiffer and may have relatively smoother surface (e.g., as compared with materials such as polycarbonate). Given that the fluid pathway of the system may be about 100 μm thick, small thickness variations in materials such as polycarbonate may shunt reagent flow along paths with higher clearance, thereby resulting in partial filling of microreactors adjacent to paths with lower clearance. Further, stiffness of glass materials may maintain a generally planar window surface under the high clamping forces of the assembled system. This in turn may result in a reagent gap with uniform clearance and therefore uniform resistance to reagent flow.

In some embodiments, it may be useful to provide opaque microreactor arrays to preclude isotropic fluorescent emission leaking between adjacent microreactors. In some embodiments, the system may include a fluorescence microscope. Suitable example microscopes include the Olympus MVX10 Macroview Fluorescent Microscope and Dino-Lite AM4115T. Assays and detection protocols may be carried out in a dark enclosure to prevent photo-bleaching. A comparison may be made of the signal intensity of dry vs. wet fluorescently labeled microreactor arrays.

In one aspect, NAPPA-ELISA may be used to detect anti-P53 autoantibody cancer biomarker. In one aspect, expressed proteins in NAPPA are appended with a tag that binds to anti-tag molecules immobilized on a surface. Proteins may remain attached to the surface of a microreactor array during subsequent blocking, washing, and labeling steps, thereby enabling immunoassays such as ELISA.

After incubating with enzymatically linked secondary antibody and subsequent washing, microreactors may be refilled with a fluorogenic substrate. Microreactors may then be resealed to isolate enzymatic reactions from each other. It may be useful to reseal the wells quickly, before the reactions proceed too far. In some embodiments, filling and sealing microreactors may take less than about 5 seconds. It may also be useful to slow the rate of a particular reaction by reducing temperature during the sealing process. Once the microreactors are sealed, they may be maintained that way for hours.

ELISA reagent concentrations may be optimized for microreactor volumes. In one example, reaction volumes are five orders of magnitude smaller in microreactors vs. standard ELISA in microplates. Therefore surface area to volume ratio is five orders of magnitude bigger. This may affect chemical reactions, especially if there is nonspecific adsorption to the surface. Reagent composition and concentration may be adjusted accordingly. For example, PCR scaled down to small reaction volumes may require addition of BSA to mitigate adsorption to the surface of the container. Typically, ELISA is an endpoint measurement. However, the present disclosure provides a system and method for performing quantitative ELISA by monitoring kinetic ELISA in real time.

In some embodiments, a method includes studying fluorescence based protein-protein interaction kinetics. For example, bound proteins (targets) may be fluorescently labeled and query molecules (probes) may be labeled with complementary fluorescent quenching molecules. Protein interaction kinetics may be monitored by signal quenching at each spot. Multiplexed monitoring of many protein-protein interactions may be simultaneously investigated in a microarray format by labeling proteins in serum samples with the fluorescent quenching molecules.

In some embodiments a system includes a fully integrated, high-throughput, automated instrument. In one aspect, the system may include high-throughput piezoelectric dispensing for filling the microreactor array. In another aspect, the system may be capable of printing 10,000 unique genes or more per microreactor array or slide.

Standard NAPPA chemistry generally involves printing spots of plasmid DNA, appended with a tag, and a printing mixture including anti-tag capture ligands, cross-linker and bovine serum albumen (BSA). In one example, whole slide surface may be first coated with the printing mixture and then DNA may be printed separately. The printing mixture may be lyophilized to preserve cross-linker functionality. Dew point may be regulated during printing to slow down evaporation allowing DNA to resolubilize printing mixture in microreactors. Slides may be incubated in a hybridization chamber after printing if necessary. Printing DNA by itself may have benefits for piezoelectric dispensing since DNA is generally much easier to dispense than protein, especially if the protein is cross-linked. The fluid path of a piezoelectric dispenser may be glass. Silicate glass surfaces immersed in water acquire negative charge, primarily through the dissociation of terminal silanol groups. DNA is also negatively charged so it may be repelled by the wet glass of a piezoelectric dispenser, thereby minimizing bio-fowling.

In one aspect, steps may be taken to prevent or reduce the occurrence of irregular spot morphology due to drying artifacts and deactivated cross-linker after drying due to labile chemistry. For example, slides may be lyophilized after coating to preserve uniform spot morphology and active crosslink chemistry. If the printing mixture does not resolubilize during printing with DNA, then the microreactor array may be spray coated with Superblock Blocking Buffer (Thermo Scientific, Rockford Ill.) or BSA or gelatin or some other suitable material to temporarily immobilize the mixture while injecting IVTT reagent and prior to sealing microreactors. Further, the printing mixture and DNA may be printed separately.

Microreactor array slides may be fabricated using isotropic wet etch chemistry. However, in some cases, isotropic wet etched slides may have irregular surface artifacts that show up in microarray scanner images and cause signal intensity variation. Accordingly, embodiments of a system may include quasi isotropic plasma etch fabrication protocols that have more uniform surface properties. Isotropic etching may produce shapes with minimal surface area. Further, surface tension may minimize surface area of a liquid. Therefore, the shape of isotropically etched microreactors may be compatible with the natural shape of a liquid. This may help distribute liquid evenly when initially filling microreactors using piezoelectric dispensing. It may further help avoid entrapping air pockets when subsequently filling microreactors with reagent. Conventional anisotropic plasma etching processes may produce sharp corners that entrap liquid or air pockets causing poor spot morphology.

Large serum sample screening runs for standard NAPPA on flat glass may be automated using a hybridization station (e.g., Tecan HS 4800 Pro) that can process about 48 standard NAPPA slides per day. However, expression may be carried out by hand using small hybridization chambers. For example, protein expression levels may not be uniformly consistent across the slide surface using the hybridization station. In one aspect, uniform protein expression may be achieved with a system according to the present disclosure. In another aspect, expressed proteins may not dry out after expression and prior to serum screening, thereby reducing or eliminating potential conformational changes and functional degradation as compared with other systems. In some embodiments, at least 10,000 protein spots may be achieved as compared with about 2,500 for standard NAPPA on flat glass. Accordingly, embodiments of an automated system may be equivalent to about four current hybridization stations processing standard NAPPA. Moreover, hybridization stations may require periodic manual injections of small volume samples and antibody labels. By comparison, the present system may include automatic sample injections and liquid handling.

Embodiments of the present disclosure may provide an open framework, allowing for the omission, substitution or addition of components in a modular fashion. Example components include solenoid valves, switching valves, tubes, fittings, vacuum source, pressure source, regulators, pressure sensors, temperature sensors, flow sensors, heaters, coolers, computers, software development system, hardware level software, digital I/O, analog I/O, cameras, graphical user interface (GUI), text based interface scripting language, and an internet interface.

Embodiments of a system may enable optimization of process parameters in a programmatic, systematic and repeatable manner. Process parameters may include microreactor shape, size and density, microreactor surface properties, fluid path thickness, valve switching sequence, flow rate, viscosity, sample volume, reagent volume, sample composition, reagent composition, pressure, vacuum, temperature, time, protocols for degassing, washing, blocking, drying, cleaning, waste disposal, and microreactor array slide handling.

In some embodiments, a system may include integrated fluorescent optical detection. For example, a system may include a high resolution CCD based camera to scan the whole of a microreactor or slide array at once. In another example, a system may be equipped with one or more consumer based cameras. Further, a system may include a separate fluorimeter reader to look at spots on the microreactor array.

In some embodiments, methods include implementing polymerase chain reaction (PCR), real-time polymerase chain reaction (real-time PCR) or quantitative real-time polymerase chain reaction (qPCR). Microreactor surfaces may be cleaned via piranha or RCA or oxygen-plasma or other means and then functionalized with epoxy-silane, e.g. 3-glycidoxypropyltrimethoxysilane (GPS) or amine functionalized with 3-aminopropyltrimethoxysilane (APTES) (APS) or poly-L-lysine (PLL) for example. PCR primers may be printed into microreactors using non-contact piezoelectric dispensers for example. The PCR primers may be attached to the surface through a combination of adsorption or electrostatic attraction for example. Microreactors may be blocked with bovine serum albumin (BSA) or Superblock Blocking Buffer in TBS (Thermo Scientific product #37535B, Rockford Ill.) by dipping or spray coating for example. The microreactor array may be inserted into the microreactor array system and PCR reaction mixture may be injected into the microreactors. The fluorescence probe signal emitted from the microreactors may be detected while undergoing thermal cycling to implement qPCR. Thermal cycling of the microreactor array system may be implemented by periodically flowing different temperature fluid through the microreactor array system or immersing the microreactor array system in an enclosure and periodically flowing different temperature fluid through the enclosure.

In some embodiments, a method includes studying label-free molecular interaction kinetics. An array of microreactors may be formed on a flexible membrane using PDMS for example. Genomic DNA may be dispensed into the microreactors using non-contact piezoelectric dispensers for example. Cell-free IVTT reagent may be injected into a reagent gap in the microreactor array system between the array of microreactors and a metalized substrate. The metalized surface may be functionalized to bind products from chemical reactions in the microreactors. Working fluid may be injected onto the sealing membrane to displace the IVTT reagent and seal the microreactors to the surface. The microreactor array system may be incubated to express target proteins from their genomic DNA. The sealing membrane may be retracted and interacting molecules (probes) may be flowed across the metalized surface. Association interactions of the probe molecules with target molecules on the metalized surface may be detected using label-free surface plasmon resonance detection for example. Washing buffer may be flowed over the metalized surface to detect disassociation of the molecular probes from the molecular targets.

Features suitable for such combinations and sub-combinations would be readily apparent to persons skilled in the art upon review of the present application as a whole. The subject matter described herein and in the recited claims intends to cover and embrace all suitable changes in technology.

The present disclosure has been described in terms of one or more preferred embodiments, and it should be appreciated that many equivalents, alternatives, variations, and modifications, aside from those expressly stated, are possible and within the scope of the disclosure.

Each reference identified in the present application is herein incorporated by reference in its entirety.

While present inventive concepts have been described with reference to particular embodiments, those of ordinary skill in the art will appreciate that various substitutions and/or other alterations may be made to the embodiments without departing from the spirit of present inventive concepts. Accordingly, the foregoing description is meant to be exemplary, and does not limit the scope of present inventive concepts.

A number of examples have been described herein. Nevertheless, it should be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the present inventive concepts.

### REFERENCES

1) Al-Housseiny, T. T., Christov, I. C. & Stone, H. A. Two-phase fluid displacement and interfacial instabilities under elastic membranes. Physical review letters 111, 034502 (2013).
2) Anderson, K. S. et al. Application of protein microarrays for multiplexed detection of antibodies to tumor antigens in breast cancer. Journal of proteome research 7, 1490-1499, doi:10.1021/pr700804c (2008).
3) Anderson, K. S. et al. Protein microarray signature of autoantibody biomarkers for the early detection of breast cancer. Journal of proteome research 10, 85-96, doi:10.1021/pr100686b (2011).
4) Angenendt, P., Kreutzberger, J., Glokler, J. & Hoheisel, J. D. 2006. Generation of High Density Protein Microarrays by Cell-Free in situ Expression of Unpurified PCR Products. Mol Cell Proteomics, 5, 1658-66.
5) Arnheim, N., Erlich, H. A., Horn, G. T., Mullis, K. B., Saiki, R. K. & Scharf, S. J. 1987. Process for Amplifying, Detecting, and/or -Cloning Nucleic Acid Sequences. US patent application. Jul. 28, 1987.
6) Blake, T. & Dobson, R. 2004. Wetting At High Capillary Numbers. 12th International Coating Science and Technology Symposium. Rochester, N.Y.
7) Blanchard, A. P., Kaiser, R. J. & Hood, L. E. 1996. High-Density Oligonucleotide Arrays. Biosensors & Bioelectronics, 11, 687-690.
8) Brenan, C. J., Roberts, D. & Hurley, J. 2009. Nanoliter High-Throughput PCR for DNA and RNA Profiling. Methods Mol Biol, 496, 161-74.
9) Brown, J. F., Silver, J. E. & Kalinina, O. V. 2000. Method of Sampling, Amplifying and Quantifying Segment of Nucleic Acid, Polymerase Chain Reaction Assembly Having Microliter-Sized Sample Chambers, and Method of Filling Assembly. US patent application. Nov. 7, 2000.
10) Brown, P. & Sahalon, T. D. 1998. Methods for Fabricating Microarrays of Biological Samples. US patent application. Sep. 15, 1998.
11) Ceroni, A. et al. Systematic analysis of the IgG antibody immune response against varicella zoster virus (VZV) using a self-assembled protein microarray. Molecular BioSystems 6, 1604-1610, doi:10.1039/c003798b (2010).
12) Chou, C. C., Chen, C. H., Lee, T. T. & Peck, K. 2004. Optimization of Probe Length and the Number of Probes per Gene for Optimal Microarray Analysis of Gene Expression. Nucleic Acids Res, 32, E99.
13) Chou, S., Krauss, P. R. & Rentstrom, P. J. 1996. Imprint Lithography with 25-Nanometer Resolution. Science, 272, 85-87.
14) de Lozar, A., Hazel, A. L. & Juel, A. Scaling properties of coating flows in rectangular channels. Physical review letters 99, 234501 (2007).
15) Du, W., Li, L., Nichols, K. P. & Ismagilov, R. F. 2009. Slipchip. Lab Chip, 9, 2286-92.
16) Dunn, P. A. & Tyrer, H. W. 1981. Quantitation of Neutrophil Phagocytosis, Using Fluorescent Latex Beads. Correlation of Microscopy and Flow Cytometry. J Lab Clin Med, 98, 374-81.
17) Eaton, S., Wiktor, P., Thirstrup, D., Lake, D. & Nagaraj, V. J. Efficacy of TRAIL treatment against HPV16 infected cervical cancer cells undergoing senescence following siRNA knockdown of E6/E7 genes. Biochemical and biophysical research communications 405, 1-6, doi:10.1016/j.bbrc.2010.12.056 (2011).
18) Engvall, E. & Perlmann, P. 1971. Enzyme-Linked Immunosorbent Assay (Elisa). Quantitative Assay of Immunoglobulin G. Immunochemistry, 8, 871-4.
19) Engvall, E. & Perlmann, P. Enzyme-linked immunosorbent assay (ELISA). Quantitative assay of immunoglobulin G. Immunochemistry 8, 871-874 (1971).
20) Ercolini, A. M. & Miller, S. D. The role of infections in autoimmune disease. Clinical and experimental immunology 155, 1-15, doi:10.1111/j.1365-2249.2008.03834.x (2009).
21) Fodor, S., Pirrung, M. C., Read, J. L. & Stryer, L. 1995. Array of Olignonucleotides on a Solid Substrate. US patent application.
22) Gibson, D. S. et al. Circulating and synovial antibody profiling of juvenile arthritis patients by nucleic acid programmable protein arrays. Arthritis research & therapy 14, R77, doi:10.1186/ar3800 (2012).
23) Gong, H., Ramalingam, N., Chen, L., Che, J., Wang, Q., Wang, Y., Yang, X., Yap, P. H. & Neo, C. H. 2006. Microfluidic Handling of PCR Solution and DNA Amplification on a Reaction Chamber Array Biochip. Biomed Microdevices, 8, 167-76.
24) Gonzalez, R. M. et al. Development and validation of sandwich ELISA microarrays with minimal assay interference. Journal of proteome research 7, 2406-2414, doi:10.1021/pr700822t (2008).
25) Gosalia, D. N. & Diamond, S. L. 2003. Printing Chemical Libraries on Microarrays for Fluid Phase Nanoliter Reactions. Proc Natl Acad Sci USA, 100, 8721-6.
26) Greener, J., Li, W., Ren, J., Voicu, D., Pakharenko, V., Tang, T. & Kumacheva, E. 2010. Rapid, Cost-Efficient Fabrication of Microfluidic Reactors In thermoplastic Polymers by Combining Photolithography and Hot Embossing. Lab Chip, 10, 522-4.
27) Groebe, D. R., Maus, M. L., Pederson, T., Clampit, J., Djuric, S., Trevillyan, J., Lin, C. W., Burns, D. J. & Warrior, U. 2003. Putting Thought to Paper: A Microarcs Protease Screen. J Biomol Screen, 8, 668-75.
28) He, M., Stoevesandt, O., Palmer, E. A., Khan, F., Ericsson, O. & Taussig, M. J. 2008. Printing Protein Arrays from DNA Arrays. Nat Methods, 5, 175-7.
29) Inglese, J., Johnson, R. L., Simeonov, A., Xia, M., Zheng, W., Austin, C. P. & Auld, D. S. 2007. High-Throughput Screening Assays for the Identification of Chemical Probes. Nat Chem Biol, 3, 466-79.
30) Jackman, R. J., Duffy, D. C., Ostuni, E., Willmore, N. D. & Whitesides, G. M. 1998. Fabricating Large Arrays of Microwells with Arbitrary Dimensions and Filling them Using Discontinuous Dewetting. Anal Chem, 70, 2280-7.
31) Jensen, O. N. Modification-specific proteomics: characterization of post-translational modifications by mass spectrometry. Current opinion in chemical biology 8, 33-41, doi:10.1016/j.cbpa.2003.12.009 (2004).
32) Juncker, D. & Pla, M. 2010. Microfluidic Microarray System and Method for the Multiplexed Analysis of Biomolecules. US Patent Application US 2010/0298163.
33) Kinpara, T., Mizuno, R., Murakami, Y., Kobayashi, M., Yamaura, S., Hasan, Q., Morita, Y., Nakano, H., Yamane, T. & Tamiya, E. 2004. A Picoliter Chamber Array for Cell-Free Protein Synthesis. J Biochem, 136, 149-54.
34) Kuruvilla, F. G., Shamji, A. F., Sternson, S. M., Hergenrother, P. J. & Schreiber, S. L. 2002. Dissecting Glucose Signalling With Diversity-oriented Synthesis and Small-Molecule Microarrays. Nature, 416, 653-7.
35) Leamon, J. H., Lee, W. L., Tartaro, K. R., Lanza, J. R., Sarkis, G. J., Dewinter, A. D., Berka, J., Weiner, M., Rothberg, J. M. & Lohman, K. L. 2003. A Massively Parallel Picotiterplate Based Platform for Discrete Picoliter-Scale Polymerase Chain Reactions. Electrophoresis, 24, 3769-77.
36) Lee, J. H., Song, Y. A., Tannenbaum, S. R. & Han, J. Increase of reaction rate and sensitivity of low-abundance enzyme assay using micro/nanofluidic preconcentration chip. Analytical chemistry 80, 3198-3204, doi:10.1021/ac800362e (2008).
37) Linton, J., Yoder, K., Hess, R., Hasan, L., Ellis, R., Kanigan, T., Friesen, K., Katz, A., Brenan, C., Morrison, T. & Garcia, J. 2010. Assay Apparatus and Method Using Microfluidic Arrays. US patent application. Mar. 23, 2010.
38) Lisi, P. J., Huang, C. W., Hoffman, R. A. & Teipel, J. W. 1982. A Fluorescence Immunoassay for Soluble Antigens Employing Flow Cytometric Detection. Clin Chim Acta, 120, 171-9.
39) Liu, H. B., Ramalingam, N., Jiang, Y., Dai, C. C., Hui, K. M. & Gong, H. Q. 2009. Rapid Distribution of a Liquid Column into a Matrix of Nanoliter Wells for Parallel Real-Time Quantitative PCR. Sensors and Actuators B: Chemical, 135, 671-677.
40) Liu, W., Chen, D., Du, W., Nichols, K. P. & Ismagilov, R. F. 2010. Slipchip for Immunoassays in Nanoliter Volumes. Anal Chem, 82, 3276-82.
41) Lu, B., Zheng, S., Quach, B. Q. & Tai, Y. C. A study of the autofluorescence of parylene materials for microTAS applications. Lab on a chip 10, 1826-1834, doi:10.1039/b924855b (2010).
42) Ma, H., Horiuchi, K. Y., Wang, Y., Kucharewicz, S. A. & Diamond, S. L. 2005. Microliter Homogenous Ultra-High Throughput Screening Microarray for Lead Discoveries and Ic50 Profiling. Assay and Drug Development Technologies, 3.
43) MacGriff, C. et al. Charge-based detection of small molecules by plasmonic-based electrochemical impedance microscopy. Analytical chemistry 85, 6682-6687, doi:10.1021/ac400475z (2013).
44) Matsubara, Y., Kobayashi, M., Morita, Y. & Tamiiya, E. 2002. Application of a Microchamber Array for DNA Amplification Using a Novel Dispensing Method. Arch Histol Cytol, 65, 481-8.
45) McCarroll, S. A. et al. Integrated detection and population-genetic analysis of SNPs and copy number variation. Nature genetics 40, 1166-1174, doi:10.1038/ng.238 (2008).
46) Metzker, M. L. Sequencing technologies—the next generation. Nature reviews. Genetics 11, 31-46, doi:10.1038/nrg2626 (2010).
47) Miersch, S. et al. Serological autoantibody profiling of type 1 diabetes by protein arrays. Journal of proteomics 94, 486-496, doi:10.1016/j.jprot.2013.10.018 (2013).
48) Montor, W. R. et al. Genome-wide study of Pseudomonas aeruginosa outer membrane protein immunogenicity using self-assembling protein microarrays. Infection and immunity 77, 4877-4886, doi:10.1128/IAI.00698-09 (2009).
49) Mullis, K., Faloona, F., Scharf, S., Saiki, R., Horn, G. & Erlich, H. 1986. Specific Enzymatic Amplification of DNA in vitro: the Polymerase Chain Reaction. Cold Spring Harb Symp Quant Biol, 51 Pt 1, 263-73.
50) Nagai, H., Murakami, Y., Morita, Y., Yokoyama, K. & Tamiya, E. 2001. Development of a Microchamber Array for Picoliter PCR. Anal Chem, 73, 1043-7.
51) Nagaraj, V. J. et al. NanoMonitor: a miniature electronic biosensor for glycan biomarker detection. Nanomedicine 5, 369-378, doi:10.2217/nnm.10.11 (2010).
52) Nagaraj, V. J., Eaton, S. & Wiktor, P. NanoProbeArrays for the analysis of ultra-low-volume protein samples using piezoelectric liquid dispensing technology. Journal of laboratory automation 16, 126-133, doi:10.1016/j.jala.2010.07.005 (2011).
53) Nagaraj, V. J., Eaton, S., Thirstrup, D. & Wiktor, P. Piezoelectric printing and probing of Lectin NanoProbeArrays for glycosylation analysis. Biochemical and biophysical research communications 375, 526-530, doi:10.1016/j.bbrc.2008.08.033 (2008). Peter Wiktor Nov. 24, 2014
54) Nikkahah, M., Stobl, J., Srinivasaraghavan, V. & Agah, M. 2013. Isotropically Etched Silicon Microarrays for Rapid Breast Cancer Cell Capture. IEEE Sensors Journal, 13, 1125-1131.
55) Northrup, M. A. & White, R. 1997. Microfabricated Reactor. US patent application. Jun. 17, 1997.
56) Okano, T., Matsuura, T., Suzuki, H. & Yomo, T. 2013. Cell-Free Protein Synthesis in a Microchamber Revealed the Presence of An Optimum Compartment Volume for High-order Reactions. ACS Synth Biol.
57) Park, J. W. 2007. Nanoliter Reactor Arrays for Antibiotic Study. Bull. Korean Chem. Soc., 28.
58) Proal, A. D., Albert, P. J. & Marshall, T. G. The human microbiome and autoimmunity. Current opinion in rheumatology 25, 234-240, doi:10.1097/BOR.0b013e32835cedbf (2013).
59) Qoronfleh, M. W. & Lindpaintner, K. Protein biomarker immunoassays opportunities and challenges. Drug Discovery World (2010).
60) Ramachandran, N. et al. Next-generation high-density self-assembling functional protein arrays. Nature methods 5, 535-538, doi:10.1038/nmeth.1210 (2008).
61) Ramachandran, N., Hainsworth, E., Bhullar, B., Eisenstein, S., Rosen, B., Lau, A. Y., Walter, J. C. & Labaer, J. 2004. Self-Assembling Protein Microarrays. Science, 305, 86-90.
62) Ramachandran, N., Srivastava, S. & Labaer, J. Applications of protein microarrays for biomarker discovery. Proteomics. Clinical applications 2, 1444-1459, doi:10.1002/prca.200800032 (2008).
63) Ramakrishnan, R., Dorris, D., Lublinsky, A., Nguyen, A., Domanus, M., Prokhorova, A., Gieser, L., Touma, E., Lockner, R., Tata, M., Zhu, X., Patterson, M., Shippy, R., Sendera, T. J. & Mazumder, A. 2002. An Assessment of Motorola Codelink Microarray Performance for Gene Expression Profiling Applications. Nucleic Acids Res, 30, E30.
64) Ramalingam, N., Liu, H. B., Dai, C. C., Jiang, Y., Wang, H., Wang, Q., K, M. H. & Gong, H. Q. 2009. Real-Time PCR Array Chip with Capillary-Driven Sample Loading and Reactor Sealing for Point-of-Care Applications. Biomed Microdevices, 11, 1007-20.
65) Ramirez, A. B. et al. Use of a single-chain antibody library for ovarian cancer biomarker discovery. Molecular & cellular proteomics: MCP 9, 1449-1460, doi:10.1074/mcp.M900496-MCP200 (2010).
66) Reddy, S., Schunk, P. & Bonnecaze, R. 2005. Dynamics of Low Capillary Number Interfaces Moving Through Sharp Features. Phys. Fluids, 17.
67) Ressine, A., Marko-Varga, G. & Laurell, T. 2007. Porous Silicon Protein Microarray Technology and Ultra-/Superhydrophobic States for Improved Bioanalytical Readout. Biotechnol Annu Rev, 13, 149-200.
68) Rissin, D. M., Kan, C. W., Campbell, T. G., Howes, S. C., Fournier, D. R., Song, L., Piech, T., Patel, P. P., Chang, L., Rivnak, A. J., Ferrell, E. P., Randall, J. D., Provuncher, G. K., Walt, D. R. & Duffy, D. C. 2010. Single-Molecule Enzyme-Linked Immunosorbent Assay Detects Serum Proteins At Subfemtomolar Concentrations. Nat Biotechnol, 28, 595-9.
69) Sakakihara, S., Araki, S., lino, R. & Noji, H. 2010. A Single-Molecule Enzymatic Assay in a Directly Accessible Femtoliter Droplet Array. Lab Chip, 10, 3355-62.
70) Sathuluri, R. R., Yamamura, S. & Tamiya, E. 2008. Microsystems Technology and Biosensing. Adv Biochem Eng Biotechnol, 109, 285-350.
71) Selgrade, M. K., Cooper, G. S., Germolec, D. R. & Heindel, J. J. Linking environmental agents and autoimmune disease: an agenda for future research. Environmental health perspectives 107 Suppl 5, 811-813 (1999).
72) Shan, X. et al. Detection of charges and molecules with self-assembled nano-oscillators. Nano letters 14, 4151-4157, doi:10.1021/n1501805e (2014).
73) Shan, X. et al. Imaging the electrocatalytic activity of single nanoparticles. Nature nanotechnology 7, 668-672, doi:10.1038/nnano.2012.134 (2012).
74) Shan, X. et al. Measuring surface charge density and particle height using surface plasmon resonance technique. Analytical chemistry 82, 234-240, doi:10.1021/ac901816z (2010).
75) Shan, X., Patel, U., Wang, S., Iglesias, R. & Tao, N. Imaging local electrochemical current via surface plasmon resonance. Science 327, 1363-1366, doi:10.1126/science.1186476 (2010).
76) Shannon, C. E. & Weaver, W. 1949. The Mathematical theory of Communication, Urbana, University of Illinois Press.
77) Shockley, W. 1950. Electrons and Holes in Semiconductors, With Applications to Transistor Electronics, New York, Van Nostrand.
78) Siuti, P., Retterer, S., Choi, C. K., Fowikes, J. & Doktycz, M. Cell Free Translation In Engineered Picoliter Volume Containers. IEEE Proceedings of the First Annual ORNL Biomed Sci Eng Cent Conf., Jun. 19, 2009.
79) Takatsy, G. 1955. The Use of Spiral Loops in Serological and Virological Micro-Methods. Acta Microbiol Acad Sci Hung, 3, 191-202.
80) Takulapalli, B. R., Qiu, J., Magee, D. M., Kahn, P., Brunner, A., Barker, K., Means, S., Miersch, S., Bian, X., Mendoza, A., Festa, F., Syal, K., Park, J. G., Labaer, J. & Wiktor, P. 2012. High Density Diffusion-Free Nanowell Arrays. J Proteome Res, 11, 4382-91.
81) Tsinberg, P., Roycroft, P., Falcovitz-Gerassi, Y. H. & Hahn, S. 2007. Microwell Biochip. US patent application. May 15, 2007.
82) Varnum, S. M., Woodbury, R. L. & Zangar, R. C. A protein microarray ELISA for screening biological fluids. Methods in molecular biology 264, 161-172, doi:10.1385/1-59259-759-9:161 (2004).
83) Victor, J., Amjad, H., Alnoor, S. & Jie Zhou, M. 2007. Methods of Sealing Micro Wells. US patent application. Dec. 25, 2007.
84) Victor, J., Huda, A. & Shivji, A. 2012. Apparatus for High Throughput Chemical Reactions. US patent application. Aug. 28, 2012.
85) Vignali, D. A. 2000. Multiplexed Particle-Based Flow Cytometric Assays. J Immunol Methods, 243, 243-55.
86) Wang, S. et al. Label-free imaging, detection, and mass measurement of single viruses by surface plasmon resonance. Proceedings of the National Academy of Sciences of the United States of America 107, 16028-16032, doi:10.1073/pnas.1005264107 (2010).
87) Wang, S., Huang, X., Shan, X., Foley, K. J. & Tao, N. Electrochemical surface plasmon resonance: basic formalism and experimental validation. Analytical chemistry 82, 935-941, doi:10.1021/ac902178f (2010).
88) Wang, W. et al. Single cells and intracellular processes studied by a plasmonic-based electrochemical impedance microscopy. Nature chemistry 3, 249-255, doi:10.1038/nchem.961 (2011).
89) Watson, J. D. & Crick, F. H. 1953a. Genetical Implications of the Structure of Deoxyribonucleic Acid. Nature, 171, 964-7.
90) Watson, J. D. & Crick, F. H. 1953b. Molecular Structure of Nucleic Acids; a Structure for Deoxyribose Nucleic Acid. Nature, 171, 737-8.
91) Wiktor, T. J., Gyorgy, E., Schlumberger, D., Sokol, F. & Koprowski, H. 1973. Antigenic Properties of Rabies Virus Components. J Immunol, 110, 269-76.
92) Wirth, T. 2008. Microwells In organic Synthesis and Catalysis, Weinheim, Wiley-Vch.
93) Woolley, A. T., Hadley, D., Landre, P., Demello, A. J., Mathies, R. A. & Northrup, M. A. 1996. Functional Integration of PCR Amplification and Capillary Electrophoresis in a Microfabricated DNA Analysis Device. Anal Chem, 68, 4081-6.
94) Wright, C. et al. Detection of multiple autoantibodies in patients with ankylosing spondylitis using nucleic acid programmable protein arrays. Molecular & cellular proteomics: MCP 11, M9 00384, doi:10.1074/mcp.M9.00384 (2012).
95) Wu, T. L. et al. Establishment of ELISA on 384-well microplate for AFP, CEA, CA 19-9, CA 15-3, CA 125, and PSA-ACT: higher sensitivity and lower reagent cost. Journal of clinical laboratory analysis 17, 241-246, doi:10.1002/jcla.10104 (2003).
96) Xia, J. L., Chen, F., Wiktor, P., Ferry, D. K. & Tao, N. J. Effect of top dielectric medium on gate capacitance of graphene field effect transistors: implications in mobility measurements and sensor applications. Nano letters 10, 5060-5064, doi:10.1021/n1103306a (2010).
97) Yalow, R. S. & Berson, S. A. 1960. Immunoassay of Endogenous Plasma Insulin In Man. J Clin Invest, 39, 1157-75.
98) Yamamoto, T., Hino, M., Kakuhata, R., Nojima, T., Shinohara, Y., Baba, Y. & Fujii, T. 2008. Evaluation of Cell-Free Protein Synthesis Using PDMS-Based Microwell Arrays. Anal Sci, 24, 243-246.
99) Yu, H., Shan, X., Wang, S., Chen, H. & Tao, N. Molecular scale origin of surface plasmon resonance biosensors. Analytical chemistry 86, 8992-8997, doi:10.1021/ac501363z (2014).
100) Zhang, C. & Xing, D. 2007. Miniaturized PCR Chips for Nucleic Acid Amplification and Analysis: Latest Advances and Future Trends. Nucleic Acids Res, 35, 4223-37.
101) Zhou, X., Cai, S., Hong, A., You, Q., Yu, P., Sheng, N., Srivannavit, O., Muranjan, S., Rouillard, J. M., Xia, Y., Zhang, X., Xiang, Q., Ganesh, R., Zhu, Q., Matejko, A., Gulari, E. & Gao, X. 2004. Microfluidic Picoarray Synthesis of Oligodeoxynucleotides and Simultaneous Assembling of Multiple DNA Sequences. Nucleic Acids Res, 32, 5409-17.
102) Ziauddin, J. & Sabatini, D. M. 2001. Microarrays of Cells Expressing Defined cDNAS. Nature, 411, 107-10.
   The citation of any document is not to be construed as an admission that it is prior art with respect to the present invention.

1. A microreactor array system comprising:
an array of microreactors;
a sealing membrane having a first surface and an opposite second surface, the sealing membrane configured to movably seal the array of microreactors;
a reagent gap providing a fluid path between the array of microreactors and the second surface of the sealing membrane when the second surface of the sealing membrane is in spaced relationship with the array of microreactors;
an injector for delivering a reagent into the reagent gap;
an applicator for directing a working fluid against the first surface of the sealing membrane; and
a detector.

2. The microreactor array system of claim 1 wherein:
a system is provided for creating a pressure differential between the reagent in the injector and the reagent gap.

3. The microreactor array system of claim 1 wherein:
the sealing membrane is impervious to liquids, providing a separation between the reagent and the working fluid.

4. The microreactor array system of claim 1 wherein:
the sealing membrane is more hydrophobic than at least a portion of a surface defining the array of microreactors.

5. The microreactor array system of claim 1 wherein:
at least one of the microreactors includes functionalizing chemicals dispersed thereon.

6. The microreactor array system of claim 1 wherein:
at least a portion of the sealing membrane includes functionalizing chemicals dispersed on the second surface.

7. The microreactor array system of claim 1 further comprising:
initiating functionalizing chemical spots and reacting functionalizing chemical spots arrayed separately onto the array of microreactors and opposing flat surface that comes into contact with the array of microreactors, the initiating functionalizing chemical spots and the reacting functionalizing chemical spots being aligned with each other thus enabling multiplexed combinatorial chemical reactions.

8. The microreactor array system of claim 1 further comprising:
a window configured with a window inlet port and a window outlet port for flowing the working fluid, and
a space between the window and microreactor array defining a gap for at least one of the reagent, the sealing membrane, and the working fluid.

9. The microreactor array system of claim 8 wherein:
a vacuum is applied using the system via at least one of the window outlet port and window inlet port to maintain at least a portion of the reagent gap open.

10. The microreactor array system of claim 1 further comprising:
a base configured with a base inlet port and a base outlet port for injecting or removing the reagent, and
a vacuum is applied to the reagent gap using the system via at least one of the base outlet port and base inlet port.

11. The microreactor array system of claim 1 wherein:
the reagent is injected into the array of microreactors via a pressure differential generated by the system.

12. The microreactor array system of claim 1 wherein:
the viscosity of the working fluid is higher than the viscosity of the reagent.

13. The microreactor array system of claim 1, wherein the detector is an optical detector, is a fluorescence detector, comprises a camera, or is a surface plasmon resonance detector.

14. A microreactor array system comprising:
a microreactor array;
a sealing membrane having a first surface and an opposite second surface, the sealing membrane configured to movably seal the array of microreactors;
a reagent gap providing a fluid path between the microreactor array and the second surface of the sealing membrane when the second surface of the sealing membrane is in spaced relationship with the array of microreactors;
an inlet for delivering a reagent into the reagent gap;
a window disposed above the first surface of the sealing membrane;
an inlet positioned in the window for directing a working fluid against the first surface of the sealing membrane; and
a detector for detecting a characteristic of the microreactor array.

15. The microreactor array system of claim 14, wherein the window is optically clear.

16. The microreactor array system of claim 14, wherein the sealing membrane comprises an optically clear material.

17. The microreactor array system of claim 14, wherein the working fluid is optically clear.

18. A method for sealing a reagent in microreactors of an array of microreactors, the method comprising:
(a) providing a microreactor array system, comprising:
(i) an array of microreactors,
(ii) a sealing membrane having a first surface and an opposite second surface, the sealing membrane configured to movably seal the array of microreactors,
(iii) a reagent gap providing a fluid path between the array of microreactors and the second surface of the sealing membrane when the second surface of the sealing membrane is in spaced relationship with the array of microreactors, and
(iv) an injector for delivering a reagent into the reagent gap;
(b) injecting the reagent into the array of microreactors using the injector;
(c) directing, using an applicator, a working fluid against the first surface of the sealing membrane to achieve a contact of at least a portion of the second surface with the array of microreactors; and
(d) detecting a characteristic of the microreactor array.

19. The method of claim 18 wherein:
the microreactor array system further comprises a system for creating a pressure differential between the reagent in the injector and the reagent gap; and
the method further comprises applying a vacuum to the reagent gap using the system via a pressure differential generated by the system.

20. The method of claim 18 wherein:
at least one of the microreactors includes functionalizing chemicals dispersed thereon.

21. The method of claim 18 wherein:
the working fluid is injected against the first surface of the sealing membrane via a pressure differential generated by the system.
